(19) United States
(12) Patent Application Publication
(10) Pub. No.: US 2007/0213308 A1
(43) Pub. Date: Sep. 13, 2007

(54) Methods, compositions, and kits for the treatment of musculoskeletal disorders and symptoms associated therewith

(76) Inventors: Jan N. Lessem, Natick, MA (US); Yanzhen Zhang, Sudbury, MA (US)

Correspondence Address: CLARK & ELBING LLP, 101 FEDERAL STREET, BOSTON, MA, 02110, US

(21) Appl. No.: 11/698,240

(22) Filed: Jan. 25, 2007

Related U.S. Application Data
(60) Provisional application No. 60/743,178, filed on Jan. 26, 2006.
(60) Provisional application No. 60/780,028, filed on Mar. 7, 2006.
(60) Provisional application No. 60/815,657, filed on Jun. 22, 2006.

(51) Int. Cl. A61K 31/573; A61K 31/519

(52) U.S. Cl. 514/171; 514/262.1

(57) ABSTRACT

The invention features methods, compositions, and kits for treating a musculoskeletal disorder, e.g., osteoarthritis, or pain, fatigue, tenderness, impairment in mobility, soft tissue swelling, or bony swelling associated therewith, by administering to a patient diagnosed with or at risk of developing such pain, fatigue, tenderness, impairment in mobility, soft tissue swelling, or bony swelling a tetra-substituted pyrimidopyrimidine, e.g., dipyridamole, or an adenosine activity upregulator, in combination with one or more additional agents. The invention further features methods, compositions, and kits for treating a patient diagnosed with or at risk of developing a musculoskeletal disorder by administering to the patient a tetra-substituted pyrimidopyrimidine or an adenosine activity upregulator in combination with one or more additional agents.

### CROSS-REFERENCE TO RELATED APPLICATIONS

This application claims benefit of U.S. Provisional Application Nos. 60/743,178, filed Jan. 26, 2006, and 60/815,657, filed Jun. 22, 2006, each of which is hereby incorporated by reference.

### FIELD OF THE INVENTION

This invention relates to the treatment of musculoskeletal disorders.

### BACKGROUND OF THE INVENTION

Musculoskeletal disorders such as arthritis are among the most frequent causes of physical disability among older adults. The three most common types of arthritis are osteoarthritis (OA), rheumatoid arthritis (RA), and gout. Osteoarthritis is the most common joint disease, with radiological evidence of its existence found in 50% of the population.

OA affects the hands, lower back, neck, and weight-bearing joints such as the knees, hips, and foot joints. The yearly incidence of OA of the hand is about 50 new cases per 1,000 for persons under age 40, rising to 65 per 1,000 for ages 40-59 and 110 per 1,000 for ages 60 and greater.

OA has been characterized as a slowly evolving degenerative disease with a multifactorial etiology that may differ depending on the joint site. OA occurs when cartilage, the tissue that cushions the ends of the bones within the joints, begins to break down and wear away. In some cases, all of the cartilage may wear away, leaving bones that rub against each other. Arthroscopic studies of early disease have shown synovitis in approximately half of those joints with cartilage damage, suggesting a localized inflammatory reaction in patients with early OA. Furthermore, numerous studies have identified an association between C-reactive protein (CRP) and OA. CRP is an acute phase response protein whose production is stimulated by cytokines, particularly interleukin-6 (IL-6). The relationship between inflammatory processes and elevation in plasma CRP and pro-inflammatory cytokines is well known. CRP has also been related to the inflammatory activity of rheumatoid arthritis.

Symptoms of OA range from stiffness and intermittent mild pain to severe joint pain and impaired biomechanical function. Symptoms can also include fatigue. Although there is no cure for most forms of OA, various therapies can help patients manage symptoms and improve their overall quality of life. Symptomatic treatment of OA traditionally involves administration of non-steroidal anti-inflammatory drugs (NSAIDs), local analgesic therapies, intra-articular corticosteroid injection, and surgery.

Treatment of OA with NSAIDs such as indomethacin, ketoprofen, ibuprofen, acetylsalicylic acid (ASA), and flurbiprofen can relieve pain by reducing local inflammation and attenuating levels of proinflammatory agents. However, long-term NSAID use is compromised by significant gastrointestinal (GI) toxicity. A large multi-center, prospective, observational study involving 1,921 patients with rheumatoid arthritis taking NSAIDs reported that 81% of patients who were hospitalized with serious GI complications had no prior GI problems. This makes it difficult for clinicians to identify patients at risk for GI side-effects. In the United States, it has been conservatively estimated that there are 107,000 annual hospitalizations for NSAID-related GI complications and 16,500 annual NSAID-related deaths among patients with RA or OA. This mortality figure is almost as high as the number of deaths due to asthma, cervical cancer and malignant melanoma combined.

Steroids are known powerful anti-inflammatory agents that have been used in treating OA. However, chronic administration of anti-inflammatory doses of steroids is also limited by well-known toxicities. For example, prolonged use of steroids has been associated with osteoporosis, high blood pressure, neurological complications, suboptimal immune response, and ocular disturbances, limiting their utility in therapeutic situations. A therapeutic agent that, for example, retained the potent anti-inflammatory effects of steroids, or the therapeutic effects of another class of drugs, while limiting the associated toxicities, would be of great benefit to patients with OA or other musculoskeletal disorders.

### SUMMARY OF THE INVENTION

The invention features methods, compositions, and kits for treating a musculoskeletal disorder, or pain, fatigue, tenderness, impairment in mobility, soft tissue swelling, or bony swelling associated with a musculoskeletal disorder, in a patient by administering to the patient in need thereof a tetra-substituted pyrimidopyrimidine or an adenosine activity upregulator, either alone or in combination with any of a number of companion compounds, including a corticosteroid, a non-steroidal anti-inflammatory drug (NSAID) (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, or tolmetin), a COX-2 inhibitor (e.g., rofecoxib, celecoxib, valdecoxib, or lumiracoxib), a biologic (e.g., abatacept, adelimumab, certolizumab, etanercept, golimumab, infliximab, rituximab, or tocilizumab), a small molecule immunomodulator (e.g., VX 702, SCIO 469, doramapimod, RO 30201195, SCIO 323, DPC 333, pranalcasan, mycophenolate, or merimepodib), a disease-modifying anti-rheumatic drug (DMARD) (e.g., methotrexate or leflunomide), a xanthine (e.g., theophylline), a non-steroidal immunophilin-dependent immunosuppressant (NsIDI) (e.g., cyclosporine, tacrolimus, ascomycin, pimecrolimus, rapamycin, or everolimus), a vitamin D analog (e.g., calcipotriene or calcipotriol), a psoralen (e.g., methoxsalen), a retinoid (e.g., acitretin or tazoretene), 5-amino salicylic acid (e.g., mesalamine, sulfasalazine, balsalazide disodium, or olsalazine sodium), hydroxychloroquine sulfate, penicillamine, or an analog of any thereof, as described herein.

Accordingly, the invention features, in one instance, a method for treating pain, fatigue, tenderness, impairment in mobility, soft tissue swelling, or bony swelling associated with a musculoskeletal disorder, e.g., osteoarthritis, by administering to a patient diagnosed with or at risk of developing such pain, fatigue, tenderness, impairment in mobility, soft tissue swelling, or bony swelling a tetra-substituted pyrimidopyrimidine, e.g., dipyridamole, or an adenosine activity upregulator, and a second drug, e.g., a corticosteroid, an NSAID, a COX-2 inhibitor, a biologic, a small molecule immunomodulator, a DMARD, a xanthine, an NsIDI, a vitamin D analog, a psoralen, a retinoid, 5-amino salicylic acid, hydroxychloroquine sulfate, or penicillamine, such that the tetra-substituted pyrimidopyrimidine or adenosine activity upregulator and the second drug are administered simultaneously or within fourteen days, ten days, five days, twenty-four hours, twelve hours, six hours, three hours, or even one hour of each other in amounts sufficient to treat the patient. Desirably, the patient experiences a reduction in pain, fatigue, tenderness, impairment in mobility, soft tissue swelling, or bony swelling subsequent to treatment, e.g., within fifty days of treatment. The reduction in pain, fatigue, tenderness, impairment in mobility, soft tissue swelling, or bony swelling can be measured using any method known in the art, e.g., a 10 cm visual analog scale, a Likert scale, the Lequesne index, the WOMAC index, the Piper Fatigue scale, or the Multidimensional Assessment of Fatigue scale. For example, an AUSCAN index that utilizes a 10 cm visual analog scale or a Likert scale may be used.

The invention also features a method for treating a musculoskeletal disorder, e.g., osteoarthritis, by administering to a patient diagnosed with or at risk of developing such a disorder a tetra-substituted pyrimidopyrimidine, e.g., dipyridamole, or an adenosine activity upregulator, and a second drug, e.g., a corticosteroid, an NSAID, a COX-2 inhibitor, a biologic, a small molecule immunomodulator, a DMARD, a xanthine, an NsIDI, a vitamin D analog, a psoralen, a retinoid, 5-amino salicylic acid, hydroxychloroquine sulfate, or penicillamine, such that the tetra-substituted pyrimidopyrimidine or adenosine activity upregulator and the second drug are administered simultaneously or within fourteen days, ten days, five days, twenty-four hours, twelve hours, six hours, three hours, or even one hour of each other in amounts sufficient to treat the patient.

In either of the foregoing instances, the tetra-substituted pyrimidopyrimidine or adenosine activity upregulator and the second drug may be administered in the same or different pharmaceutical formulations. When the second drug is a corticosteroid, the tetra-substituted pyrimidopyrimidine or adenosine activity upregulator may be administered in any useful dosage, e.g., 0.5-800 mg/day or 18-600 mg/day, in combination with a useful corticosteroid dosage, e.g., 0.1-1500 mg/day, 0.5-30 mg/day, or 0.5-10 mg/day. Compounds used in the methods of the invention may be formulated for, e.g., topical or systemic administration, and may be formulated in high, moderate, or low dosages.

In addition, a third drug, e.g., a corticosteroid, an NSAID, a COX-2 inhibitor, a biologic, a small molecule immunomodulator, a DMARD, a xanthine, an NsIDI, a vitamin D analog, a psoralen, a retinoid, 5-amino salicylic acid, hydroxychloroquine sulfate, or penicillamine may be administered to the patient such that the tetra-substituted pyrimidopyrimidine or adenosine activity upregulator, the second drug, and the third drug are administered simultaneously or within fourteen days, ten days, five days, twenty-four hours, twelve hours, six hours, three hours, or even one hour of each other in amounts sufficient to treat the patient.

The invention further features a kit that includes: (i) a composition containing a tetra-substituted pyrimidopyrimidine or an adenosine activity upregulator and a second drug, e.g., a corticosteroid, an NSAID, a COX-2 inhibitor, a biologic, a small molecule immunomodulator, a DMARD, a xanthine, an NsIDI, a vitamin D analog, a psoralen, a retinoid, 5-amino salicylic acid, hydroxychloroquine sulfate, or penicillamine; and (ii) instructions for administering the composition to a patient diagnosed with or at risk of developing a musculoskeletal disorder, or pain, fatigue, tenderness, impairment in mobility, soft tissue swelling, or bony swelling associated therewith.

In addition, the invention features a kit that includes: (i) a tetra-substituted pyrimidopyrimidine or an adenosine activity upregulator; (ii) a second drug, e.g., a corticosteroid, an NSAID, a COX-2 inhibitor, a biologic, a small molecule immunomodulator, a DMARD, a xanthine, an NsIDI, a vitamin D analog, a psoralen, a retinoid, 5-amino salicylic acid, hydroxychloroquine sulfate, and penicillamine; and (iii) instructions for administering the tetra-substituted pyrimidopyrimidine or adenosine activity upregulator and the second drug to a patient diagnosed with or at risk of developing a musculoskeletal disorder, or pain, fatigue, tenderness, impairment in mobility, soft tissue swelling, or bony swelling associated therewith.

The invention also features a kit that includes: (i) a tetra-substituted pyrimidopyrimidine or an adenosine activity upregulator; (ii) a second drug, e.g., a corticosteroid, an NSAID, a COX-2 inhibitor, a biologic, a small molecule immunomodulator, a DMARD, a xanthine, an NsIDI, a vitamin D analog, a psoralen, a retinoid, 5-amino salicylic acid, hydroxychloroquine sulfate, and penicillamine; (iii) a third drug, e.g., a corticosteroid, an NSAID, a COX-2 inhibitor, a biologic, a small molecule immunomodulator, a DMARD, a xanthine, an NsIDI, a vitamin D analog, a psoralen, a retinoid, 5-amino salicylic acid, hydroxychloroquine sulfate, or penicillamine; and (iv) instructions for administering the tetra-substituted pyrimidopyrimidine or adenosine activity upregulator, the second drug, and the third drug to a patient diagnosed with or at risk of developing a musculoskeletal disorder, or pain, fatigue, tenderness, impairment in mobility, soft tissue swelling, or bony swelling associated therewith.

The invention further features a kit that includes: (i) a drug, e.g., a corticosteroid, an NSAID, a COX-2 inhibitor, a biologic, a small molecule immunomodulator, a DMARD, a xanthine, an NsIDI, a vitamin D analog, a psoralen, a retinoid, 5-amino salicylic acid, hydroxychloroquine sulfate, or penicillamine; and (ii) instructions for administering a tetra-substituted pyrimidopyrimidine or adenosine activity upregulator and the drug to a patient diagnosed with or at risk of developing a musculoskeletal disorder, or pain, fatigue, tenderness, impairment in mobility, soft tissue swelling, or bony swelling associated therewith.

The invention additionally features a kit that includes: (i) a tetra-substituted pyrimidopyrimidine or an adenosine activity upregulator; and (ii) instructions for administering the tetra-substituted pyrimidopyrimidine or adenosine activity upregulator and a second drug, e.g., a corticosteroid (e.g., prednisolone), an NSAID, a COX-2 inhibitor, a biologic, a small molecule immunomodulator, a DMARD, a xanthine, an NsIDI, a vitamin D analog, a psoralen, a retinoid, 5-amino salicylic acid, hydroxychloroquine sulfate, or penicillamine to a patient diagnosed with or at risk of developing a musculoskeletal disorder, e.g., osteoarthritis, or pain, fatigue, tenderness, impairment in mobility, soft tissue swelling, or bony swelling associated therewith.

Tetra-substituted pyrimidopyrimidines useful in the methods, compositions, and kits of the invention include, e.g., mopidamole, dipyridamole, dipyridamole monoacetate, 2,6-disubstituted 4,8-dibenzylaminopyrimido[5,4-d]pyrimidines, 1-((2,7-bis(2-methyl-4-morpholinyl)-6-phenyl-4-pteridinyl)(2-hydroxyethyl)amino)-2-propanol, asasantin, 2,6-di-(2,2-dimethyl-1,3-dioxolan-4-yl)-methoxy-4,8-di-piperidinopyrimidopyrimidine, 2,6-bis-(2,3-dimethyoxypropoxy)-4,8-di-piperidinopyrimidopyrimidine, 2,6-bis[N,N-di(2-methoxy)ethyl]-4,6-di-piperidinopyrimidopyrimidine, and 2,6-bis(diethanolamino)-4,8-di-4-methoxybenzylaminopyrimidopyrimidine.

Corticosteroids useful in the methods, compositions, and kits of the invention include, e.g., algestone, 6-alpha-fluoroprednisolone, 6-alpha-methylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, flupredidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21 (beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin. For example, one useful combination of the invention includes dipyridamole and prednisolone.

In any of the methods, compositions, and kits of the invention, analogs of certain compounds may be employed in lieu of the compounds themselves. Analogs of tetra-substituted pyrimidopyrimidines and other compounds are described herein. Structural analogs of a compound (e.g, prednisolone) or class of compound (e.g., corticosteroid) do not need to have the same activity as the compound or class to which it is related. Thus, a non-steroidal immunophilin-dependent immunosuppressant analog does not necessarily act as an immunosuppressant.

Desirably, the methods, compositions, and kits of the invention have increased effectiveness, safety, tolerability, or satisfaction of treatment of a patient suffering from or at risk of suffering from a musculoskeletal disorder, or pain, fatigue, tenderness, impairment in mobility, soft tissue swelling, or bony swelling associated therewith, as compared to methods and compositions using each component of the combination individually.

By “corticosteroid” is meant any naturally occurring or synthetic compound characterized by a hydrogenated cyclopentanoperhydrophenanthrene ring system. Naturally occurring corticosteroids are generally produced by the adrenal cortex. Synthetic corticosteroids may be halogenated. Exemplary corticosteroids are described herein.

By “tetra-substituted pyrimidopyrimidine” is meant a compound having the formula (I): 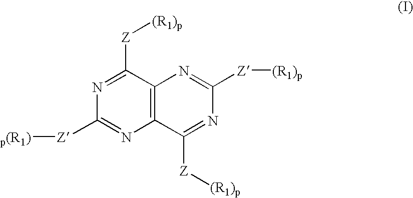
wherein each Z and each Z′ is, independently, N, O, C, 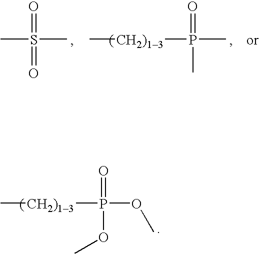
When Z or Z′ is O or 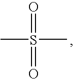
then p=1, when Z or Z′ is N, 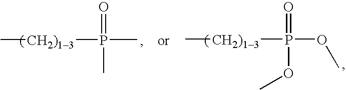
then p=2, and when Z or Z′ is C, then p=3. In formula (I), each R₁ is, independently, X, OH, N-alkyl (wherein the alkyl group has 1 to 20, more preferably 1-5, carbon atoms); a branched or unbranched alkyl group having 1 to 20, more preferably 1-5, carbon atoms; or a heterocycle, as defined herein. Alternatively, when p>1, two R₁ groups from a common Z or Z′ atom, in combination with each other, may represent —(CY₂)ₖ— in which k is an integer between 4 and 6, inclusive. Each X is, independently, Y, CY₃, C(CY₃)₃, CY₂CY₃, (CY₂)₁₋₅OY, substituted or unsubstituted cycloalkane of the structure CₙY₂ₙ₋₁, wherein n=3-7, inclusive. Each Y is, independently, H, F, Cl, Br, or I. In one embodiment, each Z is the same moiety, each Z′ is the same moiety, and Z and Z′ are different moieties.

Particularly useful tetra-substituted pyrimidopyrimidines for use in the methods, kits, and compositions of the invention are dipyridamole (also known as 2,6-bis(diethanolamino)-4,8-dipiperidinopyrimido(5,4-d)pyrimidine); 2,6-disubstituted 4,8-dibenzylaminopyrimido[5,4-d]pyrimidines; mopidamole; dipyridamole monoacetate; R-E 244 (1-((2,7-bis(2-methyl-4-morpholinyl)-6-phenyl-4-pteridinyl)(2-hydroxyethyl)amino)-2-propanol); TX-3301 (asasantin); NU3026 (2,6-di-(2,2-dimethyl-1,3-dioxolan-4-yl)-methoxy-4,8-di-piperidinopyrimidopyrimidine); NU3059 (2,6-bis-(2,3-dimethyoxypropoxy)-4,8-di-piperidinopyrimidopyrimidine); NU3060 (2,6-bis[N,N-di(2-methoxy)ethyl]-4,6-di-piperidinopyrimidopyrimidine); and NU3076 (2,6-bis(diethanolamino)-4,8-di-4-methoxybenzylaminopyrimidopyrimidine). Other tetra-substituted pyrimidopyrimidines are described in U.S. Pat. Nos. 3,031,450 and 4,963,541, hereby incorporated by reference.

By “adenosine activity upregulator” is meant adenosine and any compounds that mimic or potentiate the physiological effects of adenosine, such as adenosine receptor agonists, adenosine transport inhibitors, adenosine kinase inhibitors, and phosphodiesterase (PDE) inhibitors, as described herein.

By “non-steroidal immunophilin-dependent immunosuppressant” or “NsIDI” is meant any non-steroidal agent that decreases proinflammatory cytokine production or secretion, binds an immunophilin, or causes a downregulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

By “small molecule immunomodulator” is meant a non-steroidal, non-NsIDI compound that decreases proinflammatory cytokine production or secretion, causes a downregulation of the proinflammatory reaction, or otherwise modulates the immune system in an immunophilin-independent manner. Exemplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharamceuticals).

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of corticosteroid formulated for administration by inhalation will differ from a low dosage of corticosteroid formulated for oral administration.

By a “high dosage” is meant at least 5% (e.g., at least 10%, 20%, 50%, 100%, 200%, or even 300%) more than the highest standard recommended dosage of a particular compound for treatment of any human disease or condition.

By a “moderate dosage” is meant a dosage between the low dosage and the high dosage.

By “treating” is meant administering or prescribing a composition for the treatment or prevention of a musculoskeletal disorder, e.g., osteoarthritis, or pain, fatigue, tenderness, impairment in mobility, soft tissue swelling, or bony swelling associated therewith.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

By “an amount sufficient” is meant the amount of a compound, in a combination of the invention, required to treat or prevent a musculoskeletal disorder in a clinically relevant manner. A sufficient amount of an active compound used to practice the present invention for therapeutic treatment of conditions caused by or contributing to a musculoskeletal disorder varies depending upon the manner of administration, the age, body weight, and general health of the patient. Ultimately, the prescribers will decide the appropriate amount and dosage regimen. Additionally, an effective amount may be that amount of compound in the combination of the invention that is safe and efficacious in the treatment of a patient having the musculoskeletal disorder over each agent alone as determined and approved by a regulary authority (such as the U.S. Food and Drug Administration).

By “more effective” is meant that a method, composition, or kit exhibits greater efficacy, is less toxic, safer, more convenient, better tolerated, or less expensive, or provides more treatment satisfaction than another method, composition, or kit with which it is being compared. Efficacy may be measured by a skilled practitioner using any standard method that is appropriate for a given indication.

By “systemic administration” is meant all nondermal routes of administration, and specifically excludes topical and transdermal routes of administration.

By “sustained release” or “controlled release” is meant that the therapeutically active component is released from the formulation at a controlled rate such that therapeutically beneficial levels (but below toxic levels), e.g., blood levels, of the component are maintained over an extended period of time ranging from, e.g., about eight to about eighteen hours, thus providing, for example, an eight-hour or an eighteen-hour dosage form.

By “musculoskeletal disorder” is meant an immune system-related disorder of the muscles, ligaments, bones, joints, cartilage, or other connective tissue. Among the most commonly-occurring musculoskeletal disorders are various forms of arthritis, e.g., osteoarthritis, rheumatoid arthritis, juvenile rheumatoid arthritis, and gout. Other musculoskeletal disorders include acquired hyperostosis syndrome, acromegaly, ankylosing spondylitis, Behcet's disease, bone diseases, bursitis, cartilage diseases, chronic fatigue syndrome, compartment syndromes, congenital hypothyroidism, congenital myopathies, dentigerous cyst, dermatomyositis, diffuse idiopathic skeletal hyperostosis, Dupuytren's contracture, eosinophilia-myalgia syndrome, fasciitis, Felty's syndrome, fibromyalgia, hallux valgus, infectious arthritis, joint diseases, Kabuki make-up syndrome, Legg-Perthes disease, lupus, Lyme disease, Melas syndrome, metabolic bone diseases, mitochondrial myopathies, mixed connective tissue disease, muscular diseases, muscular dystrophies, musculoskeletal abnormalities, musculoskeletal diseases, myositis, myositis ossificans, necrotizing fasciitis, neurogenic arthropathy, osteitis deformans, osteochondritis, osteomalacia, osteomyelitis, osteonecrosis, osteoporosis, Paget's disease, Pierre Robin syndrome, polymyalgia rheumatica, polymyositis, postpoliomyelitis syndrome, pseudogout, psoriatic arthritis, reactive arthritis, Reiter disease, relapsing polychondritis, renal osteodystrophy, rhabdomyolysis, rheumatic diseases, rheumatic fever, scleroderma, Sever's disease (calceneal apophysitis), Sjögren's syndrome, spinal diseases, spinal stenosis, Still's disease, synovitis, temporomandibular joint disorders, tendinopathy, tennis elbow, tenosynovitis, Tietze's syndrome, and Wegener's granulomatosis.

By “Group A musculoskeletal disorder” is meant arthritis (e.g., osteoarthritis, rheumatoid arthritis, juvenile rheumatoid arthritis, or gout), ankylosing spondylitis, Behcet's disease, bursitis, dermatomyositis, fasciitis, fibromyalgia, lupus, myositis, myositis ossificans, necrotizing fasciitis, polymyalgia rheumatica, psoriatic arthritis, relapsing polychondritis, rheumatic fever, scleroderma, Sjögren's syndrome, Still's disease, or Wegener's granulomatosis.

By “Group B musculoskeletal disorder” is meant an immune system-related disorder of the muscles, ligaments, bones, joints, cartilage, or other connective tissue that is not a Group A musculoskeletal disorder. Exemplary Group B musculoskeletal disorders are acquired hyperostosis syndrome, acromegaly, chronic fatigue syndrome, congenital hypothyroidism, dentigerous cyst, diffuse idiopathic skeletal hyperostosis, Dupuytren's contracture, eosinophilia-myalgia syndrome, Felty's syndrome, hallux valgus, Kabuki make-up syndrome, Legg-Perthes disease, Lyme disease, Melas syndrome, neurogenic arthropathy, osteitis deformans, osteochondritis, osteomalacia, osteomyelitis, osteonecrosis, osteoporosis, Paget's disease, Pierre Robin syndrome, polymyositis, postpoliomyelitis syndrome, pseudogout, Reiter disease, renal osteodystrophy, rhabdomyolysis, Sever's disease (calceneal apophysitis), spinal stenosis, synovitis, tendinopathy, tennis elbow, tenosynovitis, and Tietze's syndrome.

In the generic descriptions of compounds used in this invention, the number of atoms of a particular type in a substituent group is generally given as a range, e.g., an alkyl group containing from 1 to 7 carbon atoms or C₁₋₇ alkyl. Reference to such a range is intended to include specific references to groups having each of the integer number of atoms within the specified range. For example, an alkyl group from 1 to 7 carbon atoms includes each of C₁, C₂, C₃, C₄, C₅, C₆, and C₇. A C₁₋₇ heteroalkyl, for example, includes from 1 to 7 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups. The C₁₋₇ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₇ alkyls include, without limitation, methyl; ethyl; n-propyl; isopropyl; cyclopropyl; cyclopropylmethyl; cyclopropylethyl; n-butyl; iso-butyl; sec-butyl; tert-butyl; cyclobutyl; cyclobutylmethyl; cyclobutylethyl; n-pentyl; cyclopentyl; cyclopentylmethyl; cyclopentylethyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-1-methylpropyl; 1-ethyl-2-methylpropyl; and cyclohexyl.

By “C₂₋₇ alkenyl” is meant a branched or unbranched hydrocarbon group containing one or more double bonds and having from 2 to 7 carbon atoms. A C₂₋₇ alkenyl may optionally include monocyclic or polycyclic rings, in which each ring desirably has from three to six members. The C₂₋₇ alkenyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₇ alkenyls include, without limitation, vinyl; allyl; 2-cyclopropyl-1-ethenyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; 2-methyl-2-pentenyl; 3-methyl-2-pentenyl; 4-methyl-2-pentenyl; 2-methyl-3-pentenyl; 3-methyl-3-pentenyl; 4-methyl-3-pentenyl; 2-methyl-4-pentenyl; 3-methyl-4-pentenyl; 1,2-dimethyl-1-propenyl; 1,2-dimethyl-1-butenyl; 1,3-dimethyl-1-butenyl; 1,2-dimethyl-2-butenyl; 1,1-dimethyl-2-butenyl; 2,3-dimethyl-2-butenyl; 2,3-dimethyl-3-butenyl; 1,3-dimethyl-3-butenyl; 1,1-dimethyl-3-butenyl and 2,2-dimethyl-3-butenyl.

By “C₂₋₇ alkynyl” is meant a branched or unbranched hydrocarbon group containing one or more triple bonds and having from 2 to 7 carbon atoms. A C₂₋₇ alkynyl may optionally include monocyclic, bicyclic, or tricyclic rings, in which each ring desirably has five or six members. The C₂₋₇ alkynyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₇ alkynyls include, without limitation, ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 4-hexynyl, 5-hexynyl; 1-methyl-2-propynyl; 1-methyl-2-butynyl; 1-methyl-3-butynyl; 2-methyl-3-butynyl; 1,2-dimethyl-3-butynyl; 2,2-dimethyl-3-butynyl; 1-methyl-2-pentynyl; 2-methyl-3-pentynyl; 1-methyl-4-pentynyl; 2-methyl-4-pentynyl; and 3-methyl-4-pentynyl.

The terms “C₂₋₆ heterocyclyl” and “heterocycle” are used interchangeably and refer to a stable 5- to 7-membered monocyclic or 7- to 14-membered bicyclic heterocyclic ring which is saturated partially unsaturated or unsaturated (aromatic), and which consists of 2 to 6 carbon atoms and 1, 2, 3 or 4 heteroatoms independently selected from the group consisting of N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom that results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 1,4,5,6-tetrahydro pyridinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, 1,4,5,6-tetrahydro pyridinyl, and tetrazolyl.

By “C₆₋₁₂ aryl” is meant an aromatic group having a ring system comprised of carbon atoms with conjugated 71 electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary subsituents include alkyl, hydroxy, alkoxy, aryloxy, sulflhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 7 to 14 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups.

By “acyl” is meant a chemical moiety with the formula R—C(O)—, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substituent of the formula —OR, wherein R is a C₆₋₁₂ aryl group.

By “halide” is meant bromine, chlorine, iodine, or fluorine.

By “fluoroalkyl” is meant an alkyl group that is substituted with a fluorine.

By “perfluoroalkyl” is meant an alkyl group consisting of only carbon and fluorine atoms.

By “carboxyalkyl” is meant a chemical moiety with the formula —(R)—COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “hydroxyalkyl” is meant a chemical moiety with the formula —(R)—OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkylthio” is meant a chemical substituent of the formula —SR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl and/or aryl groups, resulting in a positive charge at the nitrogen atom.

The term “pharmaceutically acceptable salt” represents those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein. As an example, by “prednisolone” is meant the free base as well as any pharmaceutically acceptable salt thereof (e.g., prednisolone acetate).

Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸F, and ³⁶Cl). Isotopically-labeled compounds can be prepared by synthesizing a compound using a readily available isotopically-labeled reagent in place of a non-isotopically-labeled reagent.

### BRIEF DESCRIPTION OF THE DRAWINGS

FIGS. 1A-1B are charts showing a comparison between fatigue measurements following administration of either placebo or a dipyridamole/prednisolone combination. Measurements were performed using a visual analog scale (VAS).

FIGS. 2A-2B are charts showing a comparison between fatigue measurements following administration of either placebo or a dipyridamole/prednisolone combination. Measurements were performed using the Multidimensional Assessment of Fatigue (MAF) scale.

Other features and advantages of the invention will be apparent from the following detailed description, and from the claims.

### DETAILED DESCRIPTION

The invention features, methods, compositions, and kits useful for the treatment of musculoskeletal disorders, or pain, fatigue, tenderness, impairment in mobility, soft tissue swelling, or bony swelling associated with such disorders. According to the invention, a musculoskeletal disorder, or associated pain, fatigue, tenderness, impairment in mobility, soft tissue swelling, or bony swelling, may be treated by administration of an effective amount of a tetra-substituted pyrimidopyrimidine or analog thereof (e.g., an adenosine activity upregulator), either alone or in combination with one or more companion compounds, including a corticosteroid, a non-steroidal anti-inflammatory drug (NSAID), a COX-2 inhibitor, a biologic, a small molecule immunomodulator, a disease-modifying anti-rheumatic drug (DMARD), a xanthine, a non-steroidal immunophilin-dependent immunosuppressant (NsIDI), a vitamin D analog, a psoralen, a retinoid, 5-amino salicylic acid, hydroxychloroquine sulfate, penicillamine, or an analog of any thereof.

In one instance, treatment of a musculoskeletal disorder, e.g., osteoarthritis, can be performed by administering a tetra-substituted pyrimidopyrimidine (or an analog thereof, e.g., an adenosine activity upregulator) and a corticosteroid, e.g., prednisolone, to a patient in need of such treatment. Similarly, it is possible to treat pain, fatigue, tenderness, impairment in mobility, soft tissue swelling, or bony swelling associated with a musculoskeletal disorder using the methods of the invention.

In some instances, each component of a combination of the invention may affect only part of a particular disease network, leading to incomplete or no effect on its own, while the combination selectively amplifies one or more therapeutic effects without recapitulating the toxicity of either component alone. For example, the combination of a tetra-substituted pyrimidopyrimidine or an adenosine activity upregulator and a corticosteroid can result in amplified anti-inflammatory or immunosuppressive effects in comparison to the administration of an effective dose of either agent alone, while resulting in significantly reduced toxicity.

Routes of administration for the various embodiments include, but are not limited to, topical, transdermal, and systemic administration (such as intravenous, intramuscular, subcutaneous, inhalation, rectal, buccal, vaginal, intrathecal, intraperitoneal, intraarticular, ophthalmic, or oral administration).

Any of the foregoing therapies may be administered with conventional pharmaceuticals useful for the treatment of musculoskeletal disorders.

Tetra-Substituted Pyrimidopyrimidines

Tetra-substituted pyrimidopyrimidines that can be used in the methods, compositions, and kits of the invention have the formula (I), as defined above. Tetra-substituted pyrimidopyrimidines that are useful in the methods, compositions, and kits of this invention include 2,6-disubstituted 4,8-dibenzylaminopyrimido[5,4-d]pyrimidines. Particularly useful tetra-substituted pyrimidopyrimidines include dipyridamole (also known as 2,6-bis(diethanolamino)-4,8-dipiperidinopyrimido(5,4-d)pyrimidine); mopidamole; dipyridamole monoacetate; R-E 244 (1-((2,7-bis(2-methyl-4-morpholinyl)-6-phenyl-4-pteridinyl)(2-hydroxyethyl)amino)-2-propanol); TX-3301 (asasantin); NU3026 (2,6-di-(2,2-dimethyl-1,3-dioxolan-4-yl)-methoxy-4,8-di-piperidinopyrimidopyrimidine); NU3059 (2,6-bis-(2,3-dimethyoxypropoxy)-4,8-di-piperidinopyrimidopyrimidine); NU3060 (2,6-bis[N,N-di(2-methoxy)ethyl]-4,6-di-piperidinopyrimidopyrimidine); and NU3076 (2,6-bis(diethanolamino)-4,8-di-4-methoxybenzylaminopyrimidopyrimidine). Other tetra-substituted pyrimidopyrimidines are described in U.S. Pat. Nos. 3,031,450 and 4,963,541.

The standard recommended dosage for dipyridamole for treating a musculoskeletal disorder, or pain, fatigue, tenderness, impairment in mobility, soft tissue swelling, or bony swelling associated therewith, is 50-400 mg/day, e.g., 90, 100, 150, 180, 200, 270, or 360 mg/day, although less (as little as 1, 10, or 25 mg/day) or more (e.g., 500, 600, 750, or 1,000 mg/day) may be administered.

Adenosine and Adenosine Activity Upregulators

Dipyridamole is an adenosine activity upregulator. If desired, another adenosine activity upregulator can be used in place of dipyridamole in the methods, compositions, and kits of the invention. Suitable adenosine activity upregulators are adenosine receptor agonists, adenosine transport inhibitors, adenosine kinase inhibitors, and phosphodiesterase (PDE) inhibitors, discussed below.

Adenosine Receptor Agonists

Examples of adenosine receptor agonists that can be employed in the methods, compositions, and kits of the invention are adenosine hemisulfate salt, adenosine amine congener solid, N⁶-(4-amino-3-iodophenyl)methyl-5′-N-methylcarboxamidoadenosine (1-AB-MECA); N-((2-methylphenyl)methyl)adenosine (Metrifudil); 2-(1-hexynyl)-N-methyladenosine (HEMADO); N-(1-methyl-2-phenylethyl)adenosine (R-PIA); N⁶-(R-4-hydroxyphenylisopropyl) adenosine (HPIA); N⁶-cyclopentyladenosine (CPA); N⁶-cyclopentyl-2-(3-phenylaminocarbonyltriazene-1-yl)adenosine (TCPA); N-((1S,trans)-2-hydroxycyclopentyl)adenosine (GR 79236); N⁶-cyclohexyladenosine (CHA); 2-chloro-N⁶-cyclopentyladenosine (CCPA); N-ethylcarboxamidoadenosine (NECA); 2-(4-(2-carboxyethyl)phenethylamino)-5′-N-ethylcarboxamidoadenosine (CGS 21680); N⁶-(3-iodobenzyl)-5′-N-methylcarboxamidoadenosine (IB-MECA); 2-(cyclohexylmethylidene hydrazino)adenosine (WRC 0470); 2-(4-(2-carboxyethyl)phenethylamino)-5′-N-ethylcarboxamidoadenosine (CGS 21680); N⁶-(2-(3,5-dimethoxyphenyl)-2-(2-methylphenyl)ethyl)adenosine (DPMA); hexynyladenosine-5′-N-ethylcarboxamide (HE-NECA); 2-[(2-aminoethyl-aminocarbonylethyl) phenylethylamino]-5′-N-ethyl-carboxamidoadenosine (APEC); 2-chloro-N⁶-(3-iodobenzyl)-5′-N-methylcarboxamidoadenosine (2-Cl-IB-MECA); 2-phenylaminoadenosine (CV 1808); 3′-Aminoadenosine-5′-uronamides; CV Therapeutics™ small molecule drugs Tecadenoson (CVT-510); Regadenoson (CVT 3146); and Carisa (CVT 3033); and Aderis Pharmaceuticals™ small drug molecules 2-[2-(4-chlorophenyl)ethoxy]adenosine (MRE 0094), 1-deoxy-1-[6-[[(iodophenyl)methyl]amino]-91H-purine-9-yl]-N-methyl-(-D-ribofuranuronamide) (CF101), Selodenoson (DTI-0009) and Binodenoson (MRE-0470). Other adenosine receptor agonists are those described or claimed in Gao et al., JPET, 298: 209-218 (2001); U.S. Pat. Nos. 5,278,150, 5,877,180, 6,232,297; U.S. Patent Application Publication No. 20050261236, and PCT Publication No. WO/9808855, incorporated herein by reference.

Adenosine Transport Inhibitors

Adenosine transport inhibitors that can be employed in the methods, compositions, and kits of the invention include 3-[1-(6,7-diethoxy-2-morpholinoquinazolin-4-yl)piperidin-4-yl]-1,6-dimethyl-2,4(1H,3H)-quinazolinedione hydrochloride (KF24345); 6-(4-nitrobenzyl)-thioinosine (NBI) and 6-(2-hydroxy-5-nitrobenzyl)-thioguanosine (NBG); 6-[4-(1-cyclohexyl-1H-tetrazol-5-yl)butoxy]-3,4-dihydro-2(1H)-quinolinone (Cilostazol); (2-amino-4,5-dimethyl-3-thienyl)-[3-(trifluoromethyl)phenyl]methanone (PD 81723); 3,7-dihydro-3-methyl-1-(5-oxohexyl)-7-propyl-1H-purine-2,6-dione (propentofylline); 6-[(4-nitrobenzyl)thio]-9-O-D-ribofuranosylpurine (nitrobenzylthioinosine) (NBMR); 3,4,5-trimethoxy-, (tetrahydro-1H-1,4-diazepine-1,4(5H)-diyl)di-3,1-propanediyl benzoic acid, ester (dilazep); hexobendine; dipyridamole; and adenosine transport inhibitors described in Fredholm, J. Neurochem. 62:563-573 (1994), Noji et al., J. Pharmacol. Exp. Ther. 300:200-205 (2002); and Crawley et al.; Neurosci. Lett. 36:169-174 (1983), each of which is incorporated herein by reference.

Adenosine Kinase Inhibitors

Adenosine kinase inhibitors are adenosine activity upregulators that can be used in the methods, compositions, and kits of the invention. Adenosine kinase inhibitors are generally described as either nucleoside-like, or nonnucleoside-like.

Nucleoside-Like Adenosine Kinase Inhibitors

Nucleoside-like adenosine kinase inhibitors that can be used in the methods, compositions, and kits of the invention include 5-iodotubercidin (5IT) and 2-diaryltubercidin analogues; 5′-deoxo-5′-deoxy-5-iodotubercidin (5′d-5IT); and 5′-deoxo-5′-aminoadenosine (NH₂dADO). Other nucleoside-like adenosine kinase inhibitors are described in McGaraughty et al., Current Topics in Medicinal Chemistry 5:43-58 (2005); Ugarkar, J. Med. Chem. 43:2883-2893 (2000); Ugarkar et al., J. Med. Chem. 43:2894-2905 (2000); Kaplan and Coyle, Eur. J. Pharmacol. 1:1-8 (1998); and Sinclair et al. Br. J. Pharmacol. 5:1037-1044 (2001), each of which is incorporated herein by reference.

Nonnucleoside-Like Adenosine Kinase Inhibitors

Nonnucleoside-like adenosine kinase inhibitors that can be used in the methods, compositions, and kits of the invention include 5-bromopyrrolopyrrolidine; 4-amino-5-(3-bromophenyl)-7-(6-morpholino-pyridin-3-yl)pyrido[2,3-d]pyrimidine (ABT-702). Other nonnucleoside-like AK inhibitors are described in McGaraughty et al., Current Topics in Medicinal Chemistry 5:43-58 (2005), Gomtsyan and Lee, Current Pharmaceutical Design 10:1093-1103 (2004); Jarvis et al. J. Pharm. Exp. Ther. 295:1156-1164 (2000); Kowaluk, et al. J. Pharm. Exp. Ther. 295:1165-1174 (2000); and German Patent Application DE 1014 A1, each of which is incorporated herein by reference.

Phosphodiesterase Inhibitors

Several isozymes of phosphodiesterases act as regulatory switches by catalyzing the degradation of cAMP to adenosine-5-monophosphate (5′-AMP). Inhibitors of phosphodiesterases can lead to an increase in cAMP levels, which in turn can lead to an increase in antiinflammatory actions.

Type I Phosphodiesterase Inhibitors

Type I PDE inhibitors that can be employed in the methods, compositions, and kits of the invention include (3-alpha,16-alpha)-eburnamenine-14-carboxylic acid ethyl ester (Vinpocetine); 18-methoxymethyl-3-isobutyl-1-methylxantine (MIMX); 1-carboxy-2,3,4,4a,4b,5,6,6a,6b,7,8,8a,8b,9,10,10a,14,16,17,17a,17b,18,19,19a,19b,20,21,21a,21b,22,23,23a-dotriacontahydro-14-hydroxy-8a,10a-bis(hydroxymethyl)-14-(3-methoxy-3-oxopropyl)-1,4,4a,6,6a,17b,19b,21b-octamethyl beta-D-glucopyranosiduronic acid (Ks-505a); cis-5,6a,7,8,9,9a-hexahydro-2-(4-(trifluoromethyl)phenylmethyl)-5-methyl-cyclopent (4,5)imidazo[2,1-b]purin-4(3H)-one (SCH 51866); and 2-o-propoxyphenyl-8-azapurine-6-one (Zaprinast). Other Type I PDE inhibitors are described in U.S. Patent Application Nos. 20040259792 and 20050075795, incorporated herein by reference.

Type II Phosphodiesterase Inhibitors

Type II PDE inhibitors that can be employed in the methods, compositions, and kits of the invention include erythro-9-(2-hydroxy-3-nonyl)adenine (EHNA); 2,3,6,7-tetrahydro-9,10-dimethoxy-3-methyl-2-((2,4,6-trimethylphenyl)imino)-4H-pyrimido(6,1-a)isoquinolin-4-one (trequinsin); ND7001 (Neuro3D Pharmaceuticals); and BAY 60-7550 (Alexis Biochemicals). Other Type II PDE inhibitors are described in U.S. Patent Application No. 20030176316, incorporated herein by reference.

Type III Phosphodiesterase Inhibitors

Type III PDE inhibitors that can be employed in the methods, compositions, and kits of the invention include 3-isobutyl-1-methylxanthine (IBMX); 6-dihydro-2-methyl-6-oxo-3,4′-bipyridine)-5-carbonitrile (milrinone); and N-cyclohexyl-4-((1,2-dihydro-2-oxo-6-quinolinyl)oxy)-N-methyl-butanamide (cilostamide). Other Type III PDE inhibitors are described in the following patents and patent applications: EP 0 653 426, EP 0 294 647, EP 0 357 788, EP 0 220 044, EP 0 326 307, EP 0 207 500, EP 0 406 958, EP 0 150 937, EP 0 075 463, EP 0 272 914, and EP 0 112 987, U.S. Pat. Nos. 4,963,561; 5,141,931, 6,897,229, and 6,156,753; U.S. Patent Application Nos. 20030158133, 20040097593, 20060030611, and 20060025463; WO 96/15117; DE 2825048; DE 2727481; DE 2847621; DE 3044568; DE 2837161; and DE 3021792, each of which is incorporated herein by reference.

Type IV Phosphodiesterase Inhibitors

Type IV PDE inhibitors that can be employed in the methods, compositions, and kits of the invention include 4-(3-cyclopentyloxy-4-methoxyphenyl)-2-pyrrolidone (rolipram) and 4-(3-butoxy-4-methoxybenzyl)-2-imidazolidinone (Ro20-1724). Other Type IV PDE inhibitors are described in the following patents, patent applications, and references: U.S. Pat. Nos. 3,892,777, 4,193,926, 4,655,074, 4,965,271, 5,096,906, 5,124,455, 5,272,153, 6,569,890, 6,953,853, 6,933,296, 6,919,353, 6,953,810, 6,949,573, 6,909,002, and 6,740,655; U.S. Patent Application Nos. 20030187052, 20030187257, 20030144300, 20030130254, 20030186974, 20030220352, 20030134876, 20040048903, 20040023945, 20040044036, 20040106641, 20040097593, 20040242643, 20040192701, 20040224971, 20040220183, 20040180900, 20040171798, 20040167199, 20040146561, 20040152754, 20040229918, 20050192336, 20050267196, 20050049258, 20060014782, 20060004003, 20060019932, 20050267196, 20050222207, 20050222207, 20060009481; PCT Publication No. WO 92/079778; and Molnar-Kimber, K. L. et al. J. Immunol., 150:295 A (1993), each of which is incorporated herein by reference.

Type V Phosphodiesterase Inhibitors

Type V PDE inhibitors that can be used in the methods, compositions, and kits of the invention include those described in U.S. Pat. Nos. 6,992,192, 6,984,641, 6,960,587, 6,943,166, 6,878,711, and 6,869,950, and U.S. Patent Application Nos. 20030144296, 20030171384, 20040029891, 20040038996, 20040186046, 20040259792, 20040087561, 20050054660, 20050042177, 20050245544, 20060009481, each of which is incorporated herein by reference.

Type VI Phosphodiesterase Inhibitors

Type VI PDE inhibitors that can be used in the methods, compositions, and kits of the invention include those described in U.S. Patent Application Nos. 20040259792, 20040248957, 20040242673, and 20040259880, each of which is incorporated herein by reference.

Type VII Phosphodiesterase Inhibitors

Type VII PDE inhibitors that can be used in the methods, compositions, and kits of the invention include those described in the following patents, patent application, and references: U.S. Pat. Nos. 6,838,559, 6,753,340, 6,617,357, and 6,852,720; U.S. Patent Application Nos. 20030186988, 20030162802, 20030191167, 20040214843, and 20060009481; PCT Publication WO 00/68230; and Martinez et al., J. Med. Chem. 43:683-689 (2000), each of which is incorporated herein by reference.

Non-Selective Phosphodiesterase Inhibitors

Non-selective PDE inhibitors that can be used in the methods, compositions, and kits of the invention include theophylline, papaverine, and ibudilast. Other PDE inhibitors that can be used in the methods, compositions, and kits of the invention are described in U.S. Pat. No. 6,953,774.

Corticosteroids

If desired, one or more corticosteroids may be administered in a method of the invention or may be formulated with a tetra-substituted pyrimidopyrimidine or analog thereof, e.g, an adenosine activity upregulator, in a composition of the invention. Our data show that dipyridamole in combination with the corticosteroid prednisolone is effective in reducing pain, fatigue, tenderness, impairment in mobility, and swelling associated with osteoarthritis without significant adverse effects; accordingly, the combination of a tetra-substituted pyrimidopyrimidine or an adenosine activity upregulator and a corticosteroid may be more effective in treating musculoskeletal disorders, e.g., osteoarthritis, or pain, fatigue, tenderness, impairment in mobility, soft tissue swelling, or bony swelling associated with such disorders, than either agent alone. Suitable corticosteroids include 11-alpha, 17-alpha,21-trihydroxypregn-4-ene-3,20-dione; 11-beta, 16-alpha, 17,21-tetrahydroxypregn-4-ene-3,20-dione; 11-beta, 16-alpha, 17,21-tetrahydroxypregn-1,4-diene-3,20-dione; 11-beta, 17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclometasone dipropionate; aldosterone; algestone; alphaderm; amadinone; amcinonide; anagestone; androstenedione; anecortave acetate; beclomethasone; beclomethasone dipropionate; beclomethasone dipropionate monohydrate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; dihydroelatericin a; domoprednate; doxibetasol; ecdysone; ecdysterone; endrysone; enoxolone; flucinolone; fludrocortisone; fludrocortisone acetate; flugestone; flumethasone; flumethasone pivalate; flumoxonide; flunisolide; fluocinolone; fluocinolone acetonide; fluocinonide; 9-fluorocortisone; fluocortolone; fluorohydroxyandrostenedione; fluorometholone; fluorometholone acetate; fluoxymesterone; fluprednidene; fluprednisolone; flurandrenolide; fluticasone; fluticasone propionate; formebolone; formestane; formocortal; gestonorone; glyderinine; halcinonide; hyrcanoside; halometasone; halopredone; haloprogesterone; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednisolamate; prednisolone; prednisolone 21-hemisuccinate; prednisolone acetate; prednisolone farnesylate; prednisolone hemisuccinate; prednisolone-21 (beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone diacetate; triamcinolone hexacetonide; trimegestone; turkesterone; and wortmannin.

Standard recommended dosages for corticosteroids are provided, e.g., in the Merck Manual of Diagnosis & Therapy (17th Ed. M H Beers et al., Merck & Co.) and Physicians' Desk Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone.

When the combinations of the invention are used for treatment in conjunction with corticosteroids, it is possible to reduce the dosage of the individual components substantially to a point significantly below the dosages which would be required to achieve the same effects by administering corticosteroids or tetra-substituted pyrimidopyrimidines (or adenosine activity upregulators) alone or by administering a combination of corticosteroids and tetra-substituted pyrimidopyrimidines or adenosine activity upregulators. For example, in a tetra-substituted pyrimidopyrimidine/corticosteroid combination, reduced dosages of the tetra-substituted pyrimidopyrimidine or the corticosteroid, in comparison with dosages appropriate for administration of either compound alone, may be effective in treating a musculoskeletal disorder, or pain, fatigue, tenderness, impairment in mobility, soft tissue swelling, or bony swelling associated therewith. Exemplary dosage ranges for such a combination are 50-400 mg/day tetra-substituted pyrimidopyrimidine, e.g., dipyridamole, or an adenosine activity upregulator, and 0.01-30 mg/day corticosteroid, e.g., prednisolone. Two or more corticosteroids can be administered in the same treatment.

Steroid Receptor Modulators

Steroid receptor modulators (e.g., antagonists and agonists) may be used as a substitute for or in addition to a corticosteroid in the methods, compositions, and kits of the invention. Thus, in one embodiment, the invention features the combination of a tetra-substituted pyrimidopyridine and a glucocorticoid receptor modulator or other steroid receptor modulator, and methods of treating musculoskeletal disorders, or pain, fatigue, tenderness, impairment in mobility, soft tissue swelling, or bony swelling associated with such disorders, therewith.

Glucocorticoid receptor modulators that may used in the methods, compositions, and kits of the invention include compounds described in U.S. Pat. Nos. 6,380,207, 6,380,223, 6,448,405, 6,506,766, and 6,570,020, U.S. Patent Application Publication Nos. 2003/0176478, 2003/0171585, 2003/0120081, 2003/0073703, 2002/015631, 2002/0147336, 2002/0107235, 2002/0103217, and 2001/0041802, and PCT Publication No. WO00/66522, each of which is hereby incorporated by reference. Other steroid receptor modulators that may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Other Compounds

Other compounds that may be used as a substitute for or in addition to a corticosteroid in the methods, compositions, and kits of the invention include A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

Non-Steroidal Anti-Inflammatory Drugs (NSAIDs)

If desired, the tetra-substituted pyrimidopyrimidines or adenosine activity upregulators of the invention may be administered in conjunction with one or more non-steroidal anti-inflammatory drugs (NSAIDs), such as naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin.

When a tetra-substituted pyrimidopyrimidine or an adenosine activity upregulator is administered in combination with acetylsalicylic acid, it is desirable that the combination be effective in treating musculoskeletal disorders, e.g., osteoarthritis, or pain, fatigue, tenderness, impairment in mobility, soft tissue swelling, or bony swelling associated with such disorders. Accordingly, the combination of a tetra-substituted pyrimidopyrimidine or tetra-substituted pyrimidopyrimidine analog (e.g., an adenosine activity upregulator) in combination with acetylsalicylic acid or its analogs may be more effective in treating musculoskeletal disorders, or associated pain, fatigue, tenderness, impairment in mobility, soft tissue swelling, or bony swelling, than either agent alone.

Acetylsalicylic acid, also known by trade name aspirin, is an acetyl derivative of salicylic acid and has the following structural formula:

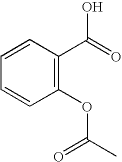

Aspirin is useful in the relief of headache and muscle and joint aches. Aspirin is also effective in reducing fever, inflammation, and swelling, and thus has been used for treatment of, e.g., osteoarthritis. Thus, the combination of a tetra-substituted pyrimidopyrimidine or analog thereof (e.g., dipyridamole, or an adenosine activity upregulator) and acetylsalicylic acid (aspirin) or analog thereof can also be administered to enhance the treatment or prevention of the disorders mentioned herein.

An NSAID may be administered in conjunction with any one of the combinations described in this application. For example, a patient suffering from a musculoskeletal disorder may be initially treated with a combination of a tetra-substituted pyrimidopyrimidine or an adenosine activity upregulator and a corticosteroid, and the patient may further be treated with an NSAID.

Dosage amounts of acetylsalicylic acid are known to those skilled in medical arts, and generally range from about 70 mg to about 350 mg per day. When a lower or a higher dose of aspirin is needed, a formulation containing dipyridamole and aspirin may contain 0-25 mg, 25-50 mg, 50-70 mg, 70-75 mg, 75-80 mg, 80-85 mg, 85-90 mg, 90-95 mg, 95-100 mg, 100-150 mg, 150-160 mg, 160-250 mg, 250-300 mg, 300-350 mg, or 350-1000 mg of aspirin.

When the combinations of the invention are used for treatment in conjunction with NSAIDs, it is possible to reduce the dosage of the individual components substantially to a point significantly below the dosages which would be required to achieve the same effects by administering NSAIDs (e.g., acetylsalicylic acid) or tetra-substituted pyrimidopyrimidines (or adenosine activity upregulators) alone or by administering a combination of NSAIDs (e.g., acetylsalicylic acid) and tetra-substituted pyrimidopyrimidines or adenosine activity upregulators.

Two or more NSAIDs can be administered in the same treatment.

Nonsteroidal Immunophilin-Dependent Immunosuppressants

In one embodiment, the invention features methods, compositions, and kits employing a tetra-substituted pyrimidopyrimidine or an adenosine activity upregulator and a non-steroidal immunophilin-dependent immunosuppressant (NsIDI), optionally with a corticosteroid or other agent described herein.

In one embodiment, the NsIDI is cyclosporine, and it is administered in an amount between 0.05 and 50 milligrams per kilogram per day (e.g., orally in an amount between 0.1 and 12 milligrams per kilogram per day). In another embodiment, the NsIDI is tacrolimus and is administered in an amount between 0.0001-20 milligrams per kilogram per day (e.g., orally in an amount between 0.01-0.2 milligrams per kilogram per day). In another embodiment, the NsIDI is rapamycin and is administered in an amount between 0.1-500 milligrams per day (e.g., at a single loading dose of 6 mg/day, followed by a 2 mg/day maintenance dose). In another embodiment, the NsIDI is everolimus, administered at a dosage of 0.75-8 mg/day. In still other embodiments, the NsIDI is pimecrolimus, administered in an amount between 0.1 and 200 milligrams per day (e.g., as a 1% cream/twice a day to treat atopic dermatitis or 60 mg a day for the treatment of psoriasis), or the NsIDI is a calcineurin-binding peptide administered in an amount and frequency sufficient to treat the patient.

When the combinations of the invention are used for treatment in conjunction with NsIDIs, it is possible to reduce the dosage of the individual components substantially to a point significantly below the dosages which would be required to achieve the same effects by administering NsIDIs or tetra-substituted pyrimidopyrimidines (or adenosine activity upregulators) alone or by administering a combination of NSAIDs and tetra-substituted pyrimidopyrimidines or adenosine activity upregulators.

Two or more NsIDIs can be administered in the same treatment.

Therapy

The invention provides therapies useful for the treatment of musculoskeletal disorders, e.g., osteoarthritis. According to the invention, a musculoskeletal disorder, or pain, fatigue, tenderness, impairment in mobility, soft tissue swelling, or bony swelling associated with such a disorder, may be treated by administration of an effective amount of a tetra-substituted pyrimidopyrimidine or analog thereof (e.g., an adenosine activity upregulator), either alone or in combination with one or more companion compounds, including a corticosteroid, an NSAID, a COX-2 inhibitor, a biologic, a small molecule immunomodulator, a DMARD, a xanthine, an NsIDI, a vitamin D analog, a psoralen, a retinoid, 5-amino salicylic acid, hydroxychloroquine sulfate, penicillamine, and an analog of any thereof.

Therapy according to the invention may be performed alone or in conjunction with another therapy and may be provided at home, a doctor's office, a clinic, a hospital's outpatient department, or a hospital. The duration of the therapy depends on the type of disease or disorder being treated, the age and condition of the patient, the stage and type of the patient's disease, and how the patient responds to the treatment. Additionally, a person having a greater risk of developing a musculoskeletal disorder (e.g., a person who is undergoing age-related hormonal changes) may receive treatment to inhibit or delay the onset of symptoms.

In particular embodiments of any of the methods of the invention, the compounds are administered within fourteen days of each other, within ten days of each other, within five days of each other, within twenty-four hours of each other, within twelve hours of each other, within six hours of each other, within three hours of each other, within one hour of each other, or simultaneously. The compounds may be formulated together as a single composition, or may be formulated and administered separately. One or both compounds may be administered in a low dosage or in a high dosage, each of which is defined herein. It may be desirable to administer to the patient one or more other compounds as well, such as corticosteroids, NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., abatacept, adelimumab, atlizumab, CDP-870, certolizumab, etanercept, golimumab, inflixamab, rituximab, and tocilizumab), small molecule immunomodulators (e.g., VX 702, SCIO 469, doramapimod, RO 30201195, SCIO 323, DPC 333, pranalcasan, mycophenolate, and merimepodib), DMARDs (e.g., methotrexate, leflunomide, minocycline, auranofin, gold sodium thiomalate, aurothioglucose, and azathioprine), xanthines (e.g., theobromine, theophylline, aminophylline, and caffeine), NsIDIs (e.g., cyclosporine, tacrolimus, pimecrolimus, and ISAtx247), vitamin D analogs (e.g., calcipotriol, tacalcitol, and maxacalcitol), psoralens (e.g., methoxsalen and trioxsalen), retinoids (e.g., tretinoin, isotretinoin, and acetretin), 5-amino salicylic acids (e.g., mesalamine, sulfasalazine, balsalazide disodium, and olsalazine sodium), hydroxychloroquine sulfate, penicillamine, or analogs thereof.

In combination therapy, the dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds.

Compounds may be administered orally in the form of tablets, capsules, elixirs or syrups, or rectally in the form of suppositories. Parenteral administration of a compound is suitably performed, for example, in the form of saline solutions or with the compound incorporated into liposomes. In cases where the compound in itself is not sufficiently soluble to be dissolved, a solubilizer such as ethanol can be applied.

Desirably, the methods, compositions, and kits of the invention are more effective than other methods, compositions, and kits.

Osteoarthritis

The methods, compositions, and kits of the invention may be used for the treatment of osteoarthritis, or pain, fatigue, tenderness, impairment in mobility, soft tissue swelling, or bony swelling associated therewith. If desired, one or more agents typically used to treat osteoarthritis may be used as a substitute for or in addition to a corticosteroid in the methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., abatacept, adelimumab, atlizumab, CDP-870, certolizumab, etanercept, golimumab, inflixamab, rituximab, and tocilizumab), small molecule immunomodulators (e.g., VX 702, SCIO 469, doramapimod, RO 30201195, SCIO 323, DPC 333, pranalcasan, mycophenolate, and merimepodib), DMARDs (e.g., methotrexate, leflunomide, minocycline, auranofin, gold sodium thiomalate, aurothioglucose, and azathioprine), xanthines (e.g., theobromine, theophylline, aminophylline, and caffeine), NsIDIs (e.g., cyclosporine, tacrolimus, pimecrolimus, and ISAtx247), vitamin D analogs (e.g., calcipotriol, tacalcitol, and maxacalcitol), psoralens (e.g., methoxsalen and trioxsalen), retinoids (e.g., tretinoin, isotretinoin, and acetretin), 5-amino salicylic acids (e.g., mesalamine, sulfasalazine, balsalazide disodium, and olsalazine sodium), hydroxychloroquine sulfate, penicillamine, or analogs thereof. Thus, in one embodiment, the invention features the combination of a tetra-substituted pyrimidopyrimidine or an adenosine activity upregulator with any of the foregoing agents, and methods and kits for the treatment of osteoarthritis, or pain, fatigue, tenderness, impairment in mobility, soft tissue swelling, or bony swelling associated therewith.

Formulation of Compositions

The administration of a combination of the invention may be by any suitable means. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each compound of the combination may be formulated in a variety of ways that are known in the art. For example, the first and second agents may be formulated together or separately. Desirably, the first and second agents are formulated together for the simultaneous or near-simultaneous administration of the agents. Such co-formulated compositions can include, for example, a tetra-substituted pyrimidopyrimidine or an adenosine activity upregulator and a corticosteroid formulated together in the same pill, capsule, liquid, or other formulation. It is to be understood that, when referring to the formulation of “tetra-substituted pyrimidopyrimidine/corticosteroid,” the formulation technology employed is also useful for the formulation of the individual agents of the combination, as well as other combinations of the invention (e.g., a tetra-substituted pyrimidopyrimidine/NSAID or tetra-substituted pyrimidopyrimidine/NsIDI). By using different formulation strategies for different agents, the pharmacokinetic profiles for each agent can be suitably matched.

The individually or separately formulated agents can be packaged together as a kit. Non-limiting examples include kits that contain, e.g., two pills, a pill and a powder, a suppository and a liquid in a vial, or two topical creams. The kit can include optional components that aid in the administration of the unit dose to patients, such as, e.g., vials for reconstituting powder forms, syringes for injection, customized IV delivery systems, or inhalers. Additionally, the unit dose kit can contain instructions for preparation and administration of the compositions.

The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Controlled and/or Extended Release Formulations

Administration of any one of the combinations of this invention, for example, a tetra-substituted pyrimidopyrimidine/corticosteroid combination in which one or both of the active agents is formulated for controlled and/or extended release, is useful, e.g., when the tetra-substituted pyrimidopyrimidine or the second agent has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; (iii) a short biological half-life; or (iv) the pharmacokinetic profile of each component must be modified to maximize the contribution of each agent, when used together, to an amount that is therapeutically effective to treat the musculoskeletal disorder, or associated pain, fatigue, tenderness, impairment in mobility, soft tissue swelling, or bony swelling. Accordingly, a sustained release formulation may be used to avoid frequent dosing that may be required in order to sustain the plasma levels of both agents at a therapeutic level. For example, in preferable oral compositions of the invention, half-life and mean residency times from ten to twenty hours for one or both agents of the combination of the invention are observed.

Many strategies can be pursued to obtain controlled and/or extended release in which the rate of release exceeds the rate of metabolism of the therapeutic compound. For example, controlled release can be obtained by the appropriate selection of formulation parameters and ingredients (e.g., appropriate controlled release compositions and coatings). Examples include single or multiple unit tablet or capsule compositions, oil solutions, suspensions, emulsions, microcapsules, microspheres, nanoparticles, patches, and liposomes. The release mechanism can be controlled such that the tetra-substituted pyrimidopyrimidine or adenosine activity upregulator and/or companion compounds (e.g., corticosteroid, NSAID, COX-2 inhibitor, biologic, small molecule immunomodulator, DMARD, xanthine, NsIDI, vitamin D analog, psoralen, retinoid, 5-amino salicylic acid, hydroxychloroquine sulfate, penicillamine, and analogs thereof, as described herein) are released at periodic intervals. The release can be simultaneous; alternatively, a delayed release of one of the agents of the combination can be effected, when the early release of one particular agent is preferred over the other.

When a tetra-substituted pyrimidopyrimidine/companion compound combination is combined with one or more additional compounds such as a corticosteroid, an NSAID, a COX-2 inhibitor, a biologic, a small molecule immunomodulator, a DMARD, a xanthine, an NsIDI, a vitamin D analog, a psoralen, a retinoid, 5-amino salicylic acid, hydroxychloroquine sulfate, or penicillamine, the release mechanism of additional compounds can also be controlled like that of the tetra-substituted pyrimidopyrimidine/companion compound combination as described herein and are similarly released at periodic intervals. The release can be simultaneous; alternatively, a delayed release of one of the agents of the combination can be effected, when the early release of one particular agent is preferred over the other.

When it is required to obtain a constant level of tetra-substituted pyrimidopyrimidine or adenosine activity upregulator in the blood, it will be advantageous to start with tetra-substituted pyrimidopyrimidine or adenosine activity upregulator in the form of pellets that enable this active substance to be released at a steady rate. For example, dipyridamole pellets can be processed together with the acetylsalicylic acid to form corresponding drug preparations. If it is intended that the acetylsalicylic acid should be released first, the dipyridamole pellets may be coated with a coating that delays the release of this active substance and the cores containing the acetylsalicylic acid coated with a coating that is soluble in gastric juices. In the case of dipyridamole pellets with a controlled release of the active substance, it is particularly advantageous to use pellets prepared according to the instructions given in U.S. Pat. No. 4,367,217.

Controlled and/or extended release formulations may include a degradable or nondegradable polymer, hydrogel, organogel, or other physical construct that modifies the bioabsorption, half-life or biodegradation of the agent. The controlled and/or extended release formulation can be a material that is painted or otherwise applied onto the afflicted site, either internally or externally. In one example, the invention provides a biodegradable bolus or implant that is surgically inserted at or near a site of interest (for example, proximal to an arthritic joint). In another instance, the controlled release formulation implant can be inserted into an organ.

Hydrogels can be used in controlled release formulations for any one of the combinations of this invention. Such polymers are formed from macromers with a polymerizable, non-degradable region that is separated by at least one degradable region. For example, the water soluble, non-degradable, region can form the central core of the macromer and have at least two degradable regions which are attached to the core, such that upon degradation, the non-degradable regions (in particular a polymerized gel) are separated, as described in U.S. Pat. No. 5,626,863. Hydrogels can include acrylates, which can be readily polymerized by several initiating systems such as eosin dye, ultraviolet or visible light. Hydrogels can also include polyethylene glycols (PEGs), which are highly hydrophilic and biocompatible. Hydrogels can also include oligoglycolic acid, which is a poly(α-hydroxy acid) that can be readily degraded by hydrolysis of the ester linkage into glycolic acid, a nontoxic metabolite. Other chain extensions can include polylacetic acid, polycaprolactone, polyorthoesters, polyanhydrides, and polypeptides. The entire network can be gelled into a biodegradable network that can be used to entrap and homogeneously disperse various combinations of the invention for delivery at a controlled rate.

Chitosan and mixtures of chitosan with carboxymethylcellulose sodium (CMC-Na) have been used as vehicles for the sustained release of drugs, e.g., as described by Inouye et al., Drug Design and Delivery 1: 297-305, 1987. Mixtures of these compounds and agents of the any one of the combinations described above, when compressed under 200 kg/cm², form a tablet from which the active agent is slowly released upon administration to a subject. The release profile can be changed by varying the ratios of chitosan, CMC-Na, and active agent(s). The tablets can also contain other additives, including lactose, CaHPO₄ dihydrate, sucrose, crystalline cellulose, or croscarmellose sodium. Several examples are given in Table 1.

TABLE 1

| Materials | Tablet components (mg) | | | | | | | | | | | |
|---|---|---|---|---|---|---|---|---|---|---|---|---|
| Active agent | 20 | 20 | 20 | 20 | 20 | 20 | 20 | 20 | 20 | 20 | 20 | 20 |
| Chitosan | 10 | 10 | 10 | 10 | 10 | 20 | 3.3 | 20 | 3.3 | 70 | 40 | 28 |
| Lactose | | 110 | | | | 220 | 36.7 | | | | | |
| CMC-Na | 60 | 60 | 60 | 60 | 60 | 120 | 20 | 120 | 20 | | 30 | 42 |
| CaHPO₄*2H₂O | | | 110 | | | | | 220 | 36.7 | 110 | 110 | 110 |
| Sucrose | 110 | | | | | | | | | | | |
| Crystalline | | | | | 110 | | | | | | | |
| Cellulose | | | | | | | | | | | | |
| Croscarmellose | | | | 110 | | | | | | | | |
| Na | | | | | | | | | | | | |

Baichwal, in U.S. Pat. No. 6,245,356, describes sustained release oral solid dosage forms that includes agglomerated particles of a therapeutically active medicament in amorphous form, a gelling agent, an ionizable gel strength enhancing agent and an inert diluent. The gelling agent can be a mixture of a xanthan gum and a locust bean gum capable of cross-linking with the xanthan gum when the gums are exposed to an environmental fluid. Preferably, the ionizable gel enhancing agent acts to enhance the strength of cross-linking between the xanthan gum and the locust bean gum and thereby prolonging the release of the medicament component of the formulation. In addition to xanthan gum and locust bean gum, acceptable gelling agents that may also be used include those gelling agents well known in the art. Examples include naturally occurring or modified naturally occurring gums such as alginates, carrageenan, pectin, guar gum, modified starch, hydroxypropylmethylcellulose, methylcellulose, and other cellulosic materials or polymers, such as, for example, sodium carboxymethylcellulose and hydroxypropyl cellulose, and mixtures of the foregoing.

In another formulation useful for the combinations of the invention, Baichwal and Staniforth, in U.S. Pat. No. 5,135,757, describe a free-flowing slow release granulation for use as a pharmaceutical excipient that includes about 20-70% or more by weight of a hydrophilic material that includes a heteropolysaccharide (such as, for example, xanthan gum or a derivative thereof) and a polysaccharide material capable of cross-linking the heteropolysaccharide (such as, for example, galactomannans, and most preferably locust bean gum) in the presence of aqueous solutions, and about 30-80% by weight of an inert pharmaceutical filler (such as, for example, lactose, dextrose, sucrose, sorbitol, xylitol, fructose or mixtures thereof). After mixing the excipient with a combination, or combination agent, of the invention, the mixture is directly compressed into solid dosage forms such as tablets. The tablets thus formed slowly release the medicament when ingested and exposed to gastric fluids. By varying the amount of excipient relative to the medicament, a slow release profile can be attained.

In another formulation useful for the combinations of the invention, Shell, in U.S. Pat. No. 5,007,790, describes sustained-release oral drug-dosage forms that release a drug in solution at a rate controlled by the solubility of the drug. The dosage form comprises a tablet or capsule that includes a plurality of particles of a dispersion of a limited solubility drug (such as, for example, prednisolone, or any other agent useful in the present invention) in a hydrophilic, water-swellable, crosslinked polymer that maintains its physical integrity over the dosing lifetime but thereafter rapidly dissolves. Once ingested, the particles swell to promote gastric retention and permit the gastric fluid to penetrate the particles, dissolve drug, and leach it from the particles, assuring that drug reaches the stomach in the solution state, which is generally better-tolerated by the stomach than solid-state drug. The programmed eventual dissolution of the polymer depends upon the nature of the polymer and the degree of crosslinking. The polymer is nonfibrillar and substantially water-soluble in its uncrosslinked state, and the degree of crosslinking is sufficient to enable the polymer to remain insoluble for the desired time period, normally at least from about four hours to eight hours or even twelve hours, with the choice depending upon the drug incorporated and the medical treatment involved. Examples of suitable crosslinked polymers that may be used in the invention are gelatin, albumin, sodium alginate, carboxymethyl cellulose, polyvinyl alcohol, and chitin. Depending upon the polymer, crosslinking may be achieved by thermal or radiation treatment or through the use of crosslinking agents such as aldehydes, polyamino acids, metal ions and the like.

Silicone microspheres for pH-controlled gastrointestinal drug delivery that are useful in the formulation of any of the combinations of the invention have been described by Carelli et al., Int. J. Pharmaceutics 179: 73-83, 1999. The microspheres so described are pH-sensitive semi-interpenetrating polymer hydrogels made of varying proportions of poly(methacrylic acid-co-methylmethacrylate) (Eudragit L100 or Eudragit S100) and crosslinked polyethylene glycol 8000 that are encapsulated into silicone microspheres in the 500-1000 μm size range.

Slow-release formulations may include a coating that is not readily water-soluble but is slowly attacked and removed by water, or through which water can slowly permeate. Thus, for example, a combination of the invention can be spray-coated with a solution of a binder under continuously fluidizing conditions, such as described by Kitamori et al. (U.S. Pat. No. 4,036,948). Water-soluble binders include pregelatinized starch (e.g., pregelatinized corn starch, pregelatinized white potato starch), pregelatinized modified starch, water-soluble celluloses (e.g. hydroxypropyl-cellulose, hydroxymethyl-cellulose, hydroxypropylmethyl-cellulose, carboxymethyl-cellulose), polyvinylpyrrolidone, polyvinyl alcohol, dextrin, gum arabicum and gelatin, and organic solvent-soluble binders, such as cellulose derivatives (e.g., cellulose acetate phthalate, hydroxypropylmethyl-cellulose phthalate, ethylcellulose).

Combinations of the invention, or a component thereof, with sustained release properties can also be formulated by spray-drying techniques. In one example, as described by Espositio et al., Pharm. Dev. Technol. 5: 267-78, 2000, prednisolone was encapsulated in methyacrylate microparticles (Eudragit RS) using a Mini Spray Dryer, model 190 (Buchi, Laboratorium Technik AG, Flawil, Germany). Optimal conditions for microparticle formation were found to be a feed (pump) rate of 0.5 mL/min of a solution containing 50 mg prednisolone in 10 mL of acetonitrile, a flow rate of nebulized air of 600 L/hr, dry air temperature heating at 80° C., and a flow rate of aspirated drying air of 28 m³/hr.

Yet another form of sustained release combinations can be prepared by microencapsulation of combination agent particles in membranes which act as microdialysis cells. In such a formulation, gastric fluid permeates the microcapsule walls and swells the microcapsule, allowing the active agent(s) to dialyze out (see, e.g., Tsuei et al., U.S. Pat. No. 5,589,194). One commercially available sustained-release system of this kind consists of microcapsules having membranes of acacia gum/gelatine/ethyl alcohol. This product is available from Eurand Limited (France) under the trade name Diffucaps™. Microcapsules so formulated might be carried in a conventional gelatine capsule or tabletted.

Other extended-release formulation examples are described in U.S. Pat. No. 5,422,123. Thus, a system for the controlled release of an active substance which is a tetra-substituted pyrimidopyrimidine such as dipyridamole, or an adenosine activity upregulator, may include (a) a deposit-core comprising an effective amount of the active substance and having defined geometric form, and (b) a support-platform applied to the deposit-core, wherein the deposit-core contains at least the active substance, and at least one member selected from the group consisting of (1) a polymeric material which swells on contact with water or aqueous liquids and a gellable polymeric material wherein the ratio of the swellable polymeric material to the gellable polymeric material is in the range 1:9 to 9:1, and (2) a single polymeric material having both swelling and gelling properties, and wherein the support-platform is an elastic support, applied to said deposit-core so that it partially covers the surface of the deposit-core and follows changes due to hydration of the deposit-core and is slowly soluble and/or slowly gellable in aqueous fluids. The support-platform may comprise polymers such as hydroxypropylmethylcellulose, plasticizers such as a glyceride, binders such as polyvinylpyrrolidone, hydrophilic agents such as lactose and silica, and/or hydrophobic agents such as magnesium stearate and glycerides. The polymer(s) typically make up 30 to 90% by weight of the support-platform, for example about 35 to 40%. Plasticizer may make up at least 2% by weight of the support-platform, for example about 15 to 20%. Binder(s), hydrophilic agent(s) and hydrophobic agent(s) typically total up to about 50% by weight of the support-platform, for example about 40 to 50%.

A controlled-release formulation of budesonide (3 mg capsules) for the treatment of inflammatory bowel disease is available from AstraZeneca (sold as Entocort™). A sustained-release formulation useful for corticosteroids is also described in U.S. Pat. No. 5,792,476, where the formulation includes 2.5-7 mg of a glucocorticoid as active substance with a regulated sustained-release such that at least 90% by weight of the glucocorticoid is released during a period of about forty to eighty minutes, starting about one to three hours after the entry of said glucocorticoid into the small intestine of the patient. To make these low dose levels of active substance possible, the active substance, i.e. the glucocorticoid, such as prednisolone or prednisone, is micronised, suitably mixed with known diluents, such as starch and lactose, and granulated with PVP (polyvinylpyrrolidone). Further, the granulate is laminated with a sustained release inner layer resistant to a pH of 6.8 and a sustained release outer layer resistant to a pH of 1.0. The inner layer is made of Eudragit® RL (copolymer of acrylic and methacrylic esters with a low content of quaternary ammonium groups) and the outer layer is made of Eudragit® L (anionic polymer synthesized from methacrylic acid and methacrylic acid methyl ester).

A bilayer tablet can be formulated for any one of the combinations described herein in which different custom granulations are made for each agent of the combination and the two agents are compressed on a bi-layer press to form a single tablet. For example, 90 mg, 180 mg, 200 mg, 360 mg, or 400 mg of dipyridamole, formulated for a controlled release, may be combined in the same tablet with 3 mg of prednisolone, which is formulated such that the t_{1/2} approximates that of dipyridamole. Examples of extended-release formulations, including those used in bilayer tablets, can be found in U.S. Pat. No. 6,548,084. Cyclodextrins are cyclic polysaccharides containing naturally-occurring D(+)-glucopyranose units in an α-(1,4) linkage. Alpha-, beta- and gamma-cyclodextrins, which contain, respectively, six, seven or eight glucopyranose units, are most commonly used, and suitable examples are described in WO91/11172, WO94/02518 and WO98/55148. Structurally, the cyclic nature of a cyclodextrin forms a torus or donut-like shape having an inner apolar or hydrophobic cavity, the secondary hydroxyl groups situated on one side of the cyclodextrin torus and the primary hydroxyl groups situated on the other. The side on which the secondary hydroxyl groups are located has a wider diameter than the side on which the primary hydroxyl groups are located. The hydrophobic nature of the cyclodextrin inner cavity allows for the inclusion of a variety of compounds. (Comprehensive Supramolecular Chemistry, Volume 3, J. L. Atwood et al., eds., Pergamon Press (1996); Cserhati, Analytical Biochemistry 225: 328-32, 1995; Husain et al., Applied Spectroscopy 46: 652-8, 1992). Cyclodextrins have been used as a delivery vehicle of various therapeutic compounds by forming inclusion complexes with various drugs that can fit into the hydrophobic cavity of the cyclodextrin or by forming non-covalent association complexes with other biologically active molecules. U.S. Pat. No. 4,727,064 describes pharmaceutical preparations consisting of a drug with substantially low water solubility and an amorphous, water-soluble cyclodextrin-based mixture in which the drug forms an inclusion complex with the cyclodextrins of the mixture.

Formation of a drug-cyclodextrin complex can modify the drug's solubility, dissolution rate, bioavailability, and/or stability properties.

Sulfobutylether-β-cyclodextrin (SBE-β-CD, commercially available from CyDex, Inc, Overland Park, Kans., and sold as CAPTISOL®) can also be used as an aid in the preparation of sustained-release formulations of agents of the combinations of the present invention. For example, a sustained-release tablet has been prepared that includes prednisolone and SBE-β-CD compressed in a hydroxypropyl methylcellulose matrix (see Rao et al., J. Pharm. Sci. 90: 807-16, 2001). In another example of the use of various cyclodextrins, EP 1109806 B1 describes cyclodextrin complexes of paroxetine, where α-, γ-, or β-cyclodextrins (including eptakis(2-6-di-O-methyl)-β-cyclodextrin, (2,3,6-tri-O-methyl)-β-cyclodextrin, monosuccinyl eptakis(2,6-di-O-methyl)-β-cyclodextrin, or 2-hydroxypropyl-β-cyclodextrin) in anhydrous or hydrated form formed complex ratios of agent to cyclodextrin of from 1:0.25 to 1:20.

Polymeric cyclodextrins have also been prepared, as described in U.S. patent application Ser. Nos. 10/021,294 and 10/021,312. The cyclodextrin polymers so formed can be useful for formulating agents of the combinations of the present invention. These multifunctional polymeric cyclodextrins are commercially available from Insert Therapeutics, Inc., Pasadena, Calif.

As an alternative to direct complexation with agents, cyclodextrins may be used as an auxiliary additive, e.g. as a carrier, diluent or solubiliser. Formulations that include cyclodextrins and other agents of the combinations of the present invention can be prepared by methods similar to the preparations of the cyclodextrin formulations described herein.

Liposomal Formulations

One or both components of any one of the combinations of the invention, or mixtures of two or more agents together, can be incorporated into liposomal carriers for administration. The liposomal carriers are composed of three general types of vesicle-forming lipid components. The first includes vesicle-forming lipids that form the bulk of the vesicle structure in the liposome. Generally, these vesicle-forming lipids include any amphipathic lipids having hydrophobic and polar head group moieties, and which (a) can form spontaneously into bilayer vesicles in water, as exemplified by phospholipids, or (b) are stably incorporated into lipid bilayers, with its hydrophobic moiety in contact with the interior, hydrophobic region of the bilayer membrane, and its polar head group moiety oriented toward the exterior, polar surface of the membrane.

The vesicle-forming lipids of this type are preferably ones having two hydrocarbon chains, typically acyl chains, and a polar head group. Included in this class are the phospholipids, such as phosphatidylcholine (PC), PE, phosphatidic acid (PA), phosphatidylinositol (PI), and sphingomyelin (SM), where the two hydrocarbon chains are typically between about 14-22 carbon atoms in length, and have varying degrees of unsaturation. The above-described lipids and phospholipids whose acyl chains have a variety of degrees of saturation can be obtained commercially, or prepared according to published methods. Other lipids that can be included in the invention are glycolipids and sterols, such as cholesterol.

The second general component includes a vesicle-forming lipid that is derivatized with a polymer chain which will form the polymer layer in the composition. The vesicle-forming lipids that can be used as the second general vesicle-forming lipid component are any of those described for the first general vesicle-forming lipid component. Vesicle forming lipids with diacyl chains, such as phospholipids, are preferred. One exemplary phospholipid is phosphatidylethanolamine (PE), which provides a reactive amino group that is convenient for coupling to the activated polymers. An exemplary PE is distearyl PE (DSPE).

The preferred polymer in the derivatized lipid is polyethyleneglycol (PEG), preferably a PEG chain having a molecular weight between 1,000-15,000 daltons, more preferably between 2,000 and 10,000 daltons, most preferably between 2,000 and 5,000 daltons. Other hydrophilic polymers that may be suitable include polyvinylpyrrolidone, polymethyloxazoline, polyethyloxazoline, polyhydroxypropyl methacrylamide, polymethacrylamide and polydimethylacrylamide, polylacetic acid, polyglycolic acid, and derivatized celluloses, such as hydroxymethylcellulose or hydroxyethylcellulose.

Additionally, block copolymers or random copolymers of these polymers, particularly including PEG segments, may be suitable. Methods for preparing lipids derivatized with hydrophilic polymers, such as PEG, are well known, e.g., as described in U.S. Pat. No. 5,013,556.

A third general vesicle-forming lipid component, which is optional, is a lipid anchor by which a targeting moiety is anchored to the liposome through a polymer chain in the anchor. Additionally, the targeting group is positioned at the distal end of the polymer chain in such a way so that the biological activity of the targeting moiety is not lost. The lipid anchor has a hydrophobic moiety which serves to anchor the lipid in the outer layer of the liposome bilayer surface, a polar head group to which the interior end of the polymer is covalently attached, and a free (exterior) polymer end which is or can be activated for covalent coupling to the targeting moiety. Methods for preparing lipid anchor molecules of these types are described below.

The lipids components used in forming the liposomes are preferably present in a molar ratio of about 70-90 percent vesicle forming lipids, 1-25 percent polymer derivatized lipid, and 0.1-5 percent lipid anchor. One exemplary formulation includes 50-70 mole percent underivatized PE, 20-40 mole percent cholesterol, 0.1-1 mole percent of a PE-PEG (3500) polymer with a chemically reactive group at its free end for coupling to a targeting moiety, 5-10 mole percent PE derivatized with PEG 3500 polymer chains, and 1 mole percent alpha-tocopherol.

The liposomes are preferably prepared to have substantially homogeneous sizes in a selected size range, typically between about 0.03 to 0.5 microns. One effective sizing method for REVs and MLVs involves extruding an aqueous suspension of the liposomes through a series of polycarbonate membranes having a selected uniform pore size in the range of 0.03 to 0.2 microns, typically 0.05, 0.08, 0.1, or 0.2 microns.

The pore size of the membrane corresponds roughly to the largest sizes of liposomes produced by extrusion through that membrane, particularly where the preparation is extruded two or more times through the same membrane. Homogenization methods are also useful for down-sizing liposomes to sizes of 100 nm or less.

The liposomal formulations of the present invention include at least one surface-active agent. Suitable surface-active agents useful for the formulation of the various combinations described herein include compounds belonging to the following classes: polyethoxylated fatty acids, PEG-fatty acid diesters, PEG-fatty acid mono-ester and di-ester mixtures, polyethylene glycol glycerol fatty acid esters, alcohol-oil transesterification products, polyglycerized fatty acids, propylene glycol fatty acid esters, mixtures of propylene glycol esters and glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene block copolymers, sorbitan fatty acid esters, lower alcohol fatty acid esters, and ionic surfactants. Commercially available examples for each class of excipient are provided below.

Polyethoxylated fatty acids may be used as excipients for the formulation of any one of the combinations described herein. Examples of commercially available polyethoxylated fatty acid monoester surfactants include: PEG 4-100 monolaurate (Crodet L series, Croda), PEG 4-100 monooleate (Crodet 0 series, Croda), PEG 4-100 monostearate (Crodet S series, Croda, and Myrj Series, Atlas/ICI), PEG 400 distearate (Cithrol 4DS series, Croda), PEG 100, 200, or 300 monolaurate (Cithrol ML series, Croda), PEG 100, 200, or 300 monooleate (Cithrol MO series, Croda), PEG 400 dioleate (Cithrol 4DO series, Croda), PEG 400-1000 monostearate (Cithrol MS series, Croda), PEG-1 stearate (Nikkol MYS-1EX, Nikko, and Coster KI, Condea), PEG-2 stearate (Nikkol MYS-2, Nikko), PEG-2 oleate (Nikkol MYO-2, Nikko), PEG-4 laurate (Mapeg® 200 ML, PPG), PEG-4 oleate (Mapeg® 200 MO, PPG), PEG-4 stearate (Kessco® PEG 200 MS, Stepan), PEG-5 stearate (Nikkol TMGS-5, Nikko), PEG-5 oleate (Nikkol TMGO-5, Nikko), PEG-6 oleate (Algon OL 60, Auschem SpA), PEG-7 oleate (Algon OL 70, Auschem SpA), PEG-6 laurate (Kessco® PEG300 ML, Stepan), PEG-7 laurate (Lauridac 7, Condea), PEG-6 stearate (Kessco® PEG300 MS, Stepan), PEG-8 laurate (Mapeg® 400 ML, PPG), PEG-8 oleate (Mapeg® 400 MO, PPG), PEG-8 stearate (Mapeg® 400 MS, PPG), PEG-9 oleate (Emulgante A9, Condea), PEG-9 stearate (Cremophor S9, BASF), PEG-10 laurate (Nikkol MYL-10, Nikko), PEG-10 oleate (Nikkol MYO-10, Nikko), PEG-12 stearate (Nikkol MYS-10, Nikko), PEG-12 laurate (Kessco® PEG 600 ML, Stepan), PEG-12 oleate (Kessco® PEG 600 MO, Stepan), PEG-12 ricinoleate (CAS # 9004-97-1), PEG-12 stearate (Mapeg® 600 MS, PPG), PEG-15 stearate (Nikkol TMGS-15, Nikko), PEG-15 oleate (Nikkol TMGO-15, Nikko), PEG-20 laurate (Kessco® PEG 1000 ML, Stepan), PEG-20 oleate (Kessco® PEG 1000 MO, Stepan), PEG-20 stearate (Mapeg® 1000 MS, PPG), PEG-25 stearate (Nikkol MYS-25, Nikko), PEG-32 laurate (Kessco® PEG 1540 ML, Stepan), PEG-32 oleate (Kessco® PEG 1540 MO, Stepan), PEG-32 stearate (Kessco® PEG 1540 MS, Stepan), PEG-30 stearate (Myrj 51), PEG-40 laurate (Crodet L40, Croda), PEG-40 oleate (Crodet 040, Croda), PEG-40 stearate (Emerest®D 2715, Henkel), PEG-45 stearate (Nikkol MYS-45, Nikko), PEG-50 stearate (Myrj 53), PEG-55 stearate (Nikkol MYS-55, Nikko), PEG-100 oleate (Crodet 0-100, Croda), PEG-100 stearate (Ariacel 165, ICI), PEG-200 oleate (Albunol 200 MO, Taiwan Surf.), PEG-400 oleate (LACTOMUL, Henkel), and PEG-600 oleate (Albunol 600 MO, Taiwan Surf.). Formulations of one or more components of any of the combinations according to the invention may include one or more of the polyethoxylated fatty acids above.

Polyethylene glycol fatty acid diesters may also be used as excipients for any of the combinations described herein. Examples of commercially available polyethylene glycol fatty acid diesters include: PEG-4 dilaurate (Mapeg® 200 DL, PPG), PEG-4 dioleate (Mapeg® 200 DO, PPG), PEG-4 distearate (Kessco® 200 DS, Stepan), PEG-6 dilaurate (Kessco® PEG 300 DL, Stepan), PEG-6 dioleate (Kessco® PEG 300 DO, Stepan), PEG-6 distearate (Kessco® PEG 300 DS, Stepan), PEG-8 dilaurate (Mapeg® 400 DL, PPG), PEG-8 dioleate (Mapeg® 400 DO, PPG), PEG-8 distearate (Mapeg® 400 DS, PPG), PEG-10 dipalmitate (Polyaldo 2PKFG), PEG-12 dilaurate (Kessco® PEG 600 DL, Stepan), PEG-12 distearate (Kessco® PEG 600 DS, Stepan), PEG-12 dioleate (Mapeg® 600 DO, PPG), PEG-20 dilaurate (Kessco® PEG 1000 DL, Stepan), PEG-20 dioleate (Kessco® PEG 1000 DO, Stepan), PEG-20 distearate (Kessco® PEG 1000 DS, Stepan), PEG-32 dilaurate (Kessco® PEG 1540 DL, Stepan), PEG-32 dioleate (Kessco® PEG 1540 DO, Stepan), PEG-32 distearate (Kessco® PEG 1540 DS, Stepan), PEG-400 dioleate (Cithrol 4DO series, Croda), and PEG-400 distearate Cithrol 4DS series, Croda). Formulations of one or more components of any of the combinations according to the invention may include one or more of the polyethylene glycol fatty acid diesters above.

PEG-fatty acid mono- and di-ester mixtures may be used as excipients for the formulation of any of the combinations described herein. Examples of commercially available PEG-fatty acid mono- and di-ester mixtures include: PEG 4-150 mono, dilaurate (Kessco® PEG 200-6000 mono, Dilaurate, Stepan), PEG 4-150 mono, dioleate (Kessco® PEG 200-6000 mono, Dioleate, Stepan), and PEG 4-150 mono, distearate (Kessco® 200-6000 mono, Distearate, Stepan). Formulations of one or more components of any of the combinations according to the invention may include one or more of the PEG-fatty acid mono- and di-ester mixtures above.

In addition, polyethylene glycol glycerol fatty acid esters may be used as excipients for the formulation of any of the combinations described herein. Examples of commercially available polyethylene glycol glycerol fatty acid esters include: PEG-20 glyceryl laurate (Tagat® L, Goldschmidt), PEG-30 glyceryl laurate (Tagat® L2, Goldschmidt), PEG-15 glyceryl laurate (Glycerox L series, Croda), PEG-40 glyceryl laurate (Glycerox L series, Croda), PEG-20 glyceryl stearate (Capmul® EMG, ABITEC), and Aldo® MS-20 KFG, Lonza), PEG-20 glyceryl oleate (Tagat® 0, Goldschmidt), and PEG-30 glyceryl oleate (Tagat® O2, Goldschmidt). Formulations of one or more components of any of the combinations according to the invention may include one or more of the polyethylene glycol glycerol fatty acid esters above.

Alcohol-oil transesterification products may also be used as excipients for the formulation of any of the combinations described herein. Examples of commercially available alcohol-oil transesterification products include: PEG-3 castor oil (Nikkol CO-3, Nikko), PEG-5, 9, and 16 castor oil (ACCONON CA series, ABITEC), PEG-20 castor oil, (Emalex C-20, Nihon Emulsion), PEG-23 castor oil (Emulgante EL23), PEG-30 castor oil (Incrocas 30, Croda), PEG-35 castor oil (Incrocas-35, Croda), PEG-38 castor oil (Emulgante EL 65, Condea), PEG-40 castor oil (Emalex C-40, Nihon Emulsion), PEG-50 castor oil (Emalex C-50, Nihon Emulsion), PEG-56 castor oil (Eumulgin® PRT 56, Pulcra SA), PEG-60 castor oil (Nikkol CO-60TX, Nikko), PEG-100 castor oil, PEG-200 castor oil (Eumulgin® PRT 200, Pulcra SA), PEG-5 hydrogenated castor oil (Nikkol HCO-5, Nikko), PEG-7 hydrogenated castor oil (Cremophor WO7, BASF), PEG-10 hydrogenated castor oil (Nikkol HCO-10, Nikko), PEG-20 hydrogenated castor oil (Nikkol HCO-20, Nikko), PEG-25 hydrogenated castor oil (Simulsol® 1292, Seppic), PEG-30 hydrogenated castor oil (Nikkol HCO-30, Nikko), PEG-40 hydrogenated castor oil (Cremophor RH 40, BASF), PEG-45 hydrogenated castor oil (Cerex ELS 450, Auschem Spa), PEG-50 hydrogenated castor oil (Emalex HC-50, Nihon Emulsion), PEG-60 hydrogenated castor oil (Nikkol HCO-60, Nikko), PEG-80 hydrogenated castor oil (Nikkol HCO-80, Nikko), PEG-100 hydrogenated castor oil (Nikkol HCO-100, Nikko), PEG-6 corn oil (Labrafil® M 2125 CS, Gattefosse), PEG-6 almond oil (Labrafil® M 1966 CS, Gattefosse), PEG-6 apricot kernel oil (Labrafil® M 1944 CS, Gattefosse), PEG-6 olive oil (Labrafil® M 1980 CS, Gattefosse), PEG-6 peanut oil (Labrafil® M 1969 CS, Gattefosse), PEG-6 hydrogenated palm kernel oil (Labrafil® M 2130 BS, Gattefosse), PEG-6 palm kernel oil (Labrafil® M 2130 CS, Gattefosse), PEG-6 triolein (Labrafil® M 2735 CS, Gattefosse), PEG-8 corn oil (Labrafil® WL 2609 BS, Gattefosse), PEG-20 corn glycerides (Crovol M40, Croda), PEG-20 almond glycerides (Crovol A40, Croda), PEG-25 trioleate (TAGAT® TO, Goldschmidt), PEG-40 palm kernel oil (Crovol PK-70), PEG-60 corn glycerides (Crovol M70, Croda), PEG-60 almond glycerides (Crovol A70, Croda), PEG-4 caprylic/capric triglyceride (Labrafac® Hydro, Gattefosse), PEG-8 caprylic/capric glycerides (Labrasol, Gattefosse), PEG-6 caprylic/capric glycerides (SOFTIGEN® 767, Huls), lauroyl macrogol-32 glyceride (GELUCIRE 44/14, Gattefosse), stearoyl macrogol glyceride (GELUCIRE 50/13, Gattefosse), mono, di, tri, tetra esters of vegetable oils and sorbitol (SorbitoGlyceride, Gattefosse), pentaerythrityl tetraisostearate (Crodamol PTIS, Croda), pentaerythrityl distearate (Albunol DS, Taiwan Surf.), pentaerythrityl tetraoleate (Liponate PO-4, Lipo Chem.), pentaerythrityl tetrastearate (Liponate PS-4, Lipo Chem.), pentaerythrityl tetracaprylate tetracaprate (Liponate PE-810, Lipo Chem.), and pentaerythrityl tetraoctanoate (Nikkol Pentarate 408, Nikko). Also included as oils in this category of surfactants are oil-soluble vitamins, such as vitamins A, D, E, K, etc. Thus, derivatives of these vitamins, such as tocopheryl PEG-1000 succinate (TPGS, available from Eastman), are also suitable surfactants. Formulations of one or more components of any of the combinations according to the invention may include one or more of the alcohol-oil transesterification products above.

Polyglycerized fatty acids may also be used as excipients for the formulation of any of the combinations described herein. Examples of commercially available polyglycerized fatty acids include: polyglyceryl-2 stearate (Nikkol DGMS, Nikko), polyglyceryl-2 oleate (Nikkol DGMO, Nikko), polyglyceryl-2 isostearate (Nikkol DGMIS, Nikko), polyglyceryl-3 oleate (Caprol® 3GO, ABITEC), polyglyceryl-4 oleate (Nikkol Tetraglyn 1-O, Nikko), polyglyceryl-4 stearate (Nikkol Tetraglyn 1-S, Nikko), polyglyceryl-6 oleate (Drewpol 6-1-O, Stepan), polyglyceryl-10 laurate (Nikkol Decaglyn 1-L, Nikko), polyglyceryl-10 oleate (Nikkol Decaglyn 1-O, Nikko), polyglyceryl-10 stearate (Nikkol Decaglyn 1-S, Nikko), polyglyceryl-6 ricinoleate (Nikkol Hexaglyn PR-15, Nikko), polyglyceryl-10 linoleate (Nikkol Decaglyn 1-LN, Nikko), polyglyceryl-6 pentaoleate (Nikkol Hexaglyn 5-O, Nikko), polyglyceryl-3 dioleate (Cremophor G032, BASF), polyglyceryl-3 distearate (Cremophor GS32, BASF), polyglyceryl-4 pentaoleate (Nikkol Tetraglyn 5-O, Nikko), polyglyceryl-6 dioleate (Caprol® 6G20, ABITEC), polyglyceryl-2 dioleate (Nikkol DGDO, Nikko), polyglyceryl-10 trioleate (Nikkol Decaglyn 3-O, Nikko), polyglyceryl-10 pentaoleate (Nikkol Decaglyn 5-O, Nikko), polyglyceryl-10 septaoleate (Nikkol Decaglyn 7-O, Nikko), polyglyceryl-10 tetraoleate (Caprol® 10G4O, ABITEC), polyglyceryl-10 decaisostearate (Nikkol Decaglyn 10-IS, Nikko), polyglyceryl-101 decaoleate (Drewpol 10-10-O, Stepan), polyglyceryl-10 mono, dioleate (Caprol® PGE 860, ABITEC), and polyglyceryl polyricinoleate (Polymuls, Henkel). Formulations of one or more components of any of the combinations according to the invention may include one or more of the polyglycerized fatty acids above.

In addition, propylene glycol fatty acid esters may be used as excipients for the formulation of any of the combinations described herein. Examples of commercially available propylene glycol fatty acid esters include: propylene glycol monocaprylate (Capryol 90, Gattefosse), propylene glycol monolaurate (Lauroglycol 90, Gattefosse), propylene glycol oleate (Lutrol OP2000, BASF), propylene glycol myristate (Mirpyl), propylene glycol monostearate (LIPO PGMS, Lipo Chem.), propylene glycol hydroxystearate, propylene glycol ricinoleate (PROPYMULS, Henkel), propylene glycol isostearate, propylene glycol monooleate (Myverol P-O6, Eastman), propylene glycol dicaprylate dicaprate (Captex® 200, ABITEC), propylene glycol dioctanoate (Captex® 800, ABITEC), propylene glycol caprylate caprate (LABRAFAC PG, Gattefosse), propylene glycol dilaurate, propylene glycol distearate (Kessco® PGDS, Stepan), propylene glycol dicaprylate (Nikkol Sefsol 228, Nikko), and propylene glycol dicaprate (Nikkol PDD, Nikko). Formulations of one or more components of any of the combinations according to the invention may include one or more of the propylene glycol fatty acid esters above.

Mixtures of propylene glycol esters and glycerol esters may also be used as excipients for the formulation of any of the combinations described herein. One preferred mixture is composed of the oleic acid esters of propylene glycol and glycerol (Arlacel 186). Examples of these surfactants include oleic (ATMOS 300, ARLACEL 186, ICI) and stearic (ATMOS150). Formulations of one or more components of any of the combinations according to the invention may include one or more of the mixtures of propylene glycol esters and glycerol esters above.

Further, mono- and diglycerides may be used as excipients for the formulation of any of the combinations described herein. Examples of commercially available mono- and diglycerides include: monopalmitolein (C16:1) (Larodan), monoelaidin (C18:1) (Larodan), monocaproin (C6)-(Larodan), monocaprylin (Larodan), monocaprin (Larodan), monolaurin (Larodan), glyceryl monomyristate (C14) (Nikkol MGM, Nikko), glyceryl monooleate (C18:1) (PECEOL, Gattefosse), glyceryl monooleate (Myverol, Eastman), glycerol monooleate/linoleate (OLICINE, Gattefosse), glycerol monolinoleate (Maisine, Gattefosse), glyceryl ricinoleate (Softigen® 701, Huls), glyceryl monolaurate (ALDO® MLD, Lonza), glycerol monopalmitate (Emalex GMS-P, Nihon), glycerol monostearate (Capmul® GMS, ABITEC), glyceryl mono- and dioleate (Capmul® GMO-K, ABITEC), glyceryl palmitic/stearic (CUTINA MD-A, ESTAGEL-G18), glyceryl acetate (Lamegin® EE, Grunau GmbH), glyceryl laurate (Imwitor® 312, Huls), glyceryl citrate/lactate/oleate/linoleate (Imwitor® 375, Huls), glyceryl caprylate (Imwitor® 308, Huls), glyceryl caprylate/caprate (Capmul® MCM, ABITEC), caprylic acid mono- and diglycerides (Imwitor® 988, Huls), caprylic/capric glycerides (Imwitor® 742, Huls), Mono-and diacetylated monoglycerides (Myvacet® 9-45, Eastman), glyceryl monostearate (Aldo® MS, Arlacel 129, ICI), lacetic acid esters of mono and diglycerides (LAMEGIN GLP, Henkel), dicaproin (C6) (Larodan), dicaprin (C10) (Larodan), dioctanoin (C8) (Larodan), dimyristin (C14) (Larodan), dipalmitin (C16) (Larodan), distearin (Larodan), glyceryl dilaurate (C12) (Capmul® GDL, ABITEC), glyceryl dioleate (Capmul® GDO, ABITEC), glycerol esters of fatty acids (GELUCIRE 39/01, Gattefosse), dipalmitolein (C16:1) (Larodan), 1,2 and 1,3-diolein (C18:1) (Larodan), dielaidin (C18:1) (Larodan), and dilinolein (C18:2) (Larodan). Formulations of one or more components of any of the combinations according to the invention may include one or more of the mono- and diglycerides above.

Sterol and sterol derivatives may also be used as excipients for the formulation of any of the combinations described herein. Examples of commercially available sterol and sterol derivatives include: cholesterol, sitosterol, lanosterol, PEG-24 cholesterol ether (Solulan C-24, Amerchol), PEG-30 cholestanol (Phytosterol GENEROL series, Henkel), PEG-25 phytosterol (Nikkol BPSH-25, Nikko), PEG-5 soyasterol (Nikkol BPS-5, Nikko), PEG-10 soyasterol (Nikkol BPS-10, Nikko), PEG-20 soyasterol (Nikkol BPS-20, Nikko), and PEG-soyasterol (Nikkol BPS-30, Nikko). Formulations of one or more components of any of the combinations according to the invention may include one or more of the sterol and sterol derivatives above.

Polyethylene glycol sorbitan fatty acid esters may also be used as excipients for the formulation of any of the combinations described herein. Examples of commercially available polyethylene glycol sorbitan fatty acid esters include: PEG-10 sorbitan laurate (Liposorb L-10, Lipo Chem.), PEG-20 sorbitan monolaurate (Tween® 20, Atlas/ICI), PEG-4 sorbitan monolaurate (Tween® 21, Atlas/ICI), PEG-80 sorbitan monolaurate (Hodag PSML-80, Calgene), PEG-6 sorbitan monolaurate (Nikkol GL-1, Nikko), PEG-20 sorbitan monopalmitate (Tween® 40, Atlas/ICI), PEG-20 sorbitan monostearate (Tweeng 60, Atlas/ICI), PEG-4 sorbitan monostearate (Tween® 61, Atlas/ICI), PEG-8 sorbitan monostearate (DACOL MSS, Condea), PEG-6 sorbitan monostearate (Nikkol TS106, Nikko), PEG-20 sorbitan tristearate (Tween® 65, Atlas/ICI), PEG-6 sorbitan tetrastearate (Nikkol GS-6, Nikko), PEG-60 sorbitan tetrastearate (Nikkol GS-460, Nikko), PEG-5 sorbitan monooleate (Tween® 81, Atlas/ICI), PEG-6 sorbitan monooleate (Nikkol TO-106, Nikko), PEG-20 sorbitan monooleate (Tween® 80, Atlas/ICI), PEG-40 sorbitan oleate (Emalex ET 8040, Nihon Emulsion), PEG-20 sorbitan trioleate (Tween® 85, Atlas/ICI), PEG-6 sorbitan tetraoleate (Nikkol GO-4, Nikko), PEG-30 sorbitan tetraoleate (Nikkol GO-430, Nikko), PEG-40 sorbitan tetraoleate (Nikkol GO-440, Nikko), PEG-20 sorbitan monoisostearate (Tween® 120, Atlas/ICI), PEG sorbitol hexaoleate (Atlas G-1086, ICI), polysorbate 80 (Tween® 80, Pharma), polysorbate 85 (Tween® 85, Pharma), polysorbate 20 (Tween® 20, Pharma), polysorbate 40 (Tween® 40, Pharma), polysorbate 60 (Tween® 60, Pharma), and PEG-6 sorbitol hexastearate (Nikkol GS-6, Nikko). Formulations of one or more components of any of the combinations according to the invention may include one or more of the polyethylene glycol sorbitan fatty acid esters above.

In addition, polyethylene glycol alkyl ethers may be used as excipients for the formulation of any of the combinations described herein. Examples of commercially available polyethylene glycol alkyl ethers include: PEG-2 oleyl ether, oleth-2 (Brij 92/93, Atlas/ICI), PEG-3 oleyl ether, oleth-3 (Volpo 3, Croda), PEG-5 oleyl ether, oleth-5 (Volpo 5, Croda), PEG-10 oleyl ether, oleth-10 (Volpo 10, Croda), PEG-20 oleyl ether, oleth-20 (Volpo 20, Croda), PEG-4 lauryl ether, laureth-4 (Brij 30, Atlas/ICI), PEG-9 lauryl ether, PEG-23 lauryl ether, laureth-23 (Brij 35, Atlas/ICI), PEG-2 cetyl ether (Brij 52, ICI), PEG-10 cetyl ether (Brij 56, ICI), PEG-20 cetyl ether (BriJ 58, ICI), PEG-2 stearyl ether (Brij 72, ICI), PEG-10 stearyl ether (Brij 76, ICI), PEG-20 stearyl ether (Brij 78, ICI), and PEG-100 stearyl ether (Brij 700, ICI). Formulations of one or more components of any of the combinations according to the invention may include one or more of the polyethylene glycol alkyl ethers above.

Sugar esters may also be used as excipients for the formulation of any of the combinations described herein. Examples of commercially available sugar esters include: sucrose distearate (SUCRO ESTER 7, Gattefosse), sucrose distearate/monostearate (SUCRO ESTER 11, Gattefosse), sucrose dipalmitate, sucrose monostearate (Crodesta F-160, Croda), sucrose monopalmitate (SUCRO ESTER 15, Gattefosse), and sucrose monolaurate (Saccharose monolaurate 1695, Mitsubisbi-Kasei). Formulations of one or more components of any of the combinations according to the invention may include one or more of the sugar esters above.

Polyethylene glycol alkyl phenols are also useful as excipients for the formulation of any of the combinations described herein. Examples of commercially available polyethylene glycol alkyl phenols include: PEG-10-100 nonylphenol series (Triton X series, Rohm & Haas) and PEG-15-100 octylphenol ether series (Triton N-series, Rohm & Haas). Formulations of one or more components of any of the combinations according to the invention may include one or more of the polyethylene glycol alkyl phenols above.

Polyoxyethylene-polyoxypropylene block copolymers may also be used as excipients for the formulation of any of the combinations described herein. These surfactants are available under various trade names, including one or more of Synperonic PE series (ICI), Pluronic® series (BASF), Lutrol (BASF), Supronic, Monolan, Pluracare, and Plurodac. The generic term for these copolymers is “poloxamer” (CAS 9003-11-6). These polymers have the formula shown below:
HO(C₂H₄O)ₐ(C₃H₆O)_{b}(C₂H₄O)ₐH
where “a” and “b” denote the number of polyoxyethylene and polyoxypropylene units, respectively. These copolymers are available in molecular weights ranging from 1000 to 15000 daltons, and with ethylene oxide/propylene oxide ratios between 0.1 and 0.8 by weight. Formulations of one or more components of any of the combinations according to the invention may include one or more of the polyoxyethylene-polyoxypropylene block copolymers above.

Polyoxyethylenes, such as PEG 300, PEG 400, and PEG 600, may be used as excipients for the formulation of any of the combinations described herein.

Sorbitan fatty acid esters may also be used as excipients for the formulation of any of the combinations described herein. Examples of commercially sorbitan fatty acid esters include: sorbitan monolaurate (Span-20, Atlas/ICI), sorbitan monopalmitate (Span-40, Atlas/ICI), sorbitan monooleate (Span-80, Atlas/ICI), sorbitan monostearate (Span-60, Atlas/ICI), sorbitan trioleate (Span-85, Atlas/ICI), sorbitan sesquioleate (Arlacel-C, ICI), sorbitan tristearate (Span-65, Atlas/ICI), sorbitan monoisostearate (Crill 6, Croda), and sorbitan sesquistearate (Nikkol SS-15, Nikko). Formulations of one or more components of any of the combinations according to the invention may include one or more of the sorbitan fatty acid esters above.

Esters of lower alcohols (C₂ to C₄) and fatty acids (C₈ to C₁₈) are suitable surfactants for use in the invention. Examples of these surfactants include: ethyl oleate (Crodamol EO, Croda), isopropyl myristate (Crodamol IPM, Croda), isopropyl palmitate (Crodamol IPP, Croda), ethyl linoleate (Nikkol VF-E, Nikko), and isopropyl linoleate (Nikkol VF-IP, Nikko). Formulations of one or more components of any of the combinations according to the invention may include one or more of the lower alcohol fatty acid esters above.

In addition, ionic surfactants may be used as excipients for the formulation of any of the combinations described herein. Examples of useful ionic surfactants include: sodium caproate, sodium caprylate, sodium caprate, sodium laurate, sodium myristate, sodium myristolate, sodium palmitate, sodium palmitoleate, sodium oleate, sodium ricinoleate, sodium linoleate, sodium linolenate, sodium stearate, sodium lauryl sulfate (dodecyl), sodium tetradecyl sulfate, sodium lauryl sarcosinate, sodium dioctyl sulfosuccinate, sodium cholate, sodium taurocholate, sodium glycocholate, sodium deoxycholate, sodium taurodeoxycholate, sodium glycodeoxycholate, sodium ursodeoxycholate, sodium chenodeoxycholate, sodium taurochenodeoxycholate, sodium glyco cheno deoxycholate, sodium cholylsarcosinate, sodium N-methyl taurocholate, egg yolk phosphatides, hydrogenated soy lecithin, dimyristoyl lecithin, lecithin, hydroxylated lecithin, lysophosphatidylcholine, cardiolipin, sphingomyelin, phosphatidylcholine, phosphatidyl ethanolamine, phosphatidic acid, phosphatidyl glycerol, phosphatidyl serine, diethanolamine, phospholipids, polyoxyethylene-10 oleyl ether phosphate, esterification products of fatty alcohols or fatty alcohol ethoxylates, with phosphoric acid or anhydride, ether carboxylates (by oxidation of terminal OH group of, fatty alcohol ethoxylates), succinylated monoglycerides, sodium stearyl fumarate, stearoyl propylene glycol hydrogen succinate, mono/diacetylated tartaric acid esters of mono- and diglycerides, citric acid esters of mono-, diglycerides, glyceryl-lacto esters of fatty acids, acyl lactylates, lactylic esters of fatty acids, sodium stearoyl-2-lactylate, sodium stearoyl lactylate, alginate salts, propylene glycol alginate, ethoxylated alkyl sulfates, alkyl benzene sulfones, α-olefin sulfonates, acyl isethionates, acyl taurates, alkyl glyceryl ether sulfonates, sodium octyl sulfosuccinate, sodium undecylenamideo-MEA-sulfosuccinate, hexadecyl triammonium bromide, decyl trimethyl ammonium bromide, cetyl trimethyl ammonium bromide, dodecyl ammonium chloride, alkyl benzyldimethylammonium salts, diisobutyl phenoxyethoxydimethyl benzylammonium salts, alkylpyridinium salts, betaines (trialkylglycine), lauryl betaine (N-lauryl,N,N-dimethylglycine), and ethoxylated amines (polyoxyethylene-15 coconut amine). For simplicity, typical counterions are provided above. It will be appreciated by one skilled in the art, however, that any bioacceptable counterion may be used. For example, although the fatty acids are shown as sodium salts, other cation counterions can also be used, such as, for example, alkali metal cations or ammonium. Formulations of one or more components of any of the combinations according to the invention may include one or more of the ionic surfactants above.

The excipients present in the formulations of the invention are present in amounts such that the carrier forms a clear, or opalescent, aqueous dispersion of the tetra-substituted pyrimidopyrimidine or adenosine activity upregulator, or the corticosteroid, or any of the combination sequestered within the liposome. The relative amount of a surface active excipient necessary for the preparation of liposomal or solid lipid nanoparticulate formulations is determined using known methodology. For example, liposomes may be prepared by a variety of techniques, such as those detailed in Szoka et al, 1980. Multilamellar vesicles (MLVs) can be formed by simple lipid-film hydration techniques. In this procedure, a mixture of liposome-forming lipids of the type detailed above dissolved in a suitable organic solvent is evaporated in a vessel to form a thin film, which is then covered by an aqueous medium. The lipid film hydrates to form MLVs, typically with sizes between about 0.1 to 10 microns.

Other established liposomal formulation techniques can be applied as needed. For example, the use of liposomes to facilitate cellular uptake is described in U.S. Pat. Nos. 4,897,355 and 4,394,448.

Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

The two compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

Thus, for compositions adapted for oral use, an oral vehicle (e.g., a capsule) containing from between 0.01% to 25% (w/w) or more of a tetra-substituted pyrimidopyrimidine or analog (e.g., an adenosine activity upregulator) and/or additional agent, preferably from between 0.01% to 10% (w/w), more preferably from between 0.05% to 4% (w/w) active agent. The capsule can be taken one to four times daily, or as needed.

For example, for dipyridamole adapted for oral administration, an oral vehicle will contain from between 0.01% to 5% (w/w), preferably from between 0.01% to 2% (w/w), more preferably from between 0.01% to 1% (w/w) dipyridamole.

Performing the methods described herein, the oral vehicle containing a compound of dipyridamole or dipyridamole analog and/or additional agent is preferably taken orally. For example, a capsule may be taken in the morning and one in the evening by a patient suffering from a musculoskeletal disorder, or pain, fatigue, tenderness, impairment in mobility, soft tissue swelling, or bony swelling related to such a disorder.

Topical Formulations

Compositions can also be adapted for topical use with a topical vehicle containing from between 0.0001% to 25% (w/w) or more of tetra-substituted pyrimidopyrimidine and/or analog (e.g., an adenosine activity upregulator) and between 0.001% to 25% (w/w) or more of another compound, e.g., a corticosteroid. In such combinations, it is preferred that the tetra-substituted pyrimidopyrimidine or adenosine activity upregulator is subjected to an extended-release mechanism.

In one combination, the tetra-substituted pyrimidopyrimidine or adenosine activity upregulator and corticosteroid may be from between, e.g., 0.0001% to 10% (w/w), or 0.0005% to 4% (w/w), active agent. The cream can be applied one to four times daily, or as needed. For example, for prednisolone adapted for topical administration, a topical vehicle will contain from between 0.01% to 5% (w/w), preferably from between 0.01% to 2% (w/w), more preferably from between 0.01% to 1% (w/w) prednisolone in combination with tetra-substituted pyrimidopyrimidine or adenosine activity upregulator, which is 0.0001% to 2% (w/w), more preferably from between 0.0005% to 1% (w/w).

Performing the methods described herein, a topical vehicle containing, e.g., a tetra-substituted pyrimidopyrimidine or an adenosine activity upregulator combined with a corticosteroid or corticosteroid analog, is preferably applied to the site of discomfort on the subject. For example, a cream may be applied to the hands of a subject suffering from osteoarthritis.

Inhalation

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

Dosages

Given the enhanced potency of the combinations of the invention, it is understood that a low dosage (as defined herein) of the tetra-substituted pyrimidopyrimidine or adenosine activity upregulator and/or the additional agents can be used. These dosages will vary depending on the health and condition of the patient. Thus, a moderate dosage or even a high dosage of one or both agents can be used.

Administration of each drug in the combination can, independently, be, e.g., one to four times daily for one day to one year, and may even be for the life of the patient. Chronic, long-term administration will be indicated in many cases.

Pain, Function, and Fatigue Indices

In order to measure the efficacy of any of the methods, compositions, or kits of the invention, a measurement index may be used. Indices that are useful in the methods, compositions, and kits of the invention include a visual analog scale (VAS), a Likert scale, the Lequesne index, the WOMAC index, the AUSCAN index, the Piper Fatigue Scale, and the Multidimensional Assessment of Fatigue (MAF) scale, each of which is well known in the art. Such indices may be used to measure pain, function, fatigue, stiffness, tenderness, impairment in mobility, soft tissue swelling, bony swelling, or other variables.

A visual analog scale (VAS) provides a measure of a one-dimensional quantity. A VAS generally utilizes a representation of distance, such as a picture of a line with hash marks drawn at regular distance intervals, e.g., ten 1-cm intervals. For example, a patient can be asked to rank a sensation of pain by choosing the spot on the line that best corresponds to the sensation of pain, where one end of the line corresponds to “no pain” (score of 0 cm) and the other end of the line corresponds to “unbearable pain” (score of 10 cm). This procedure provides a simple and rapid approach to obtaining quantitative information about how the patient is experiencing pain. VAS scales can also be used, e.g., to measure fatigue. VAS scales and their use are described, e.g., in U.S. Pat. Nos. 6,709,406 and 6,432,937.

A Likert scale similarly provides a measure of a one-dimensional quantity. Generally, a Likert scale has discrete integer values ranging from a low value (e.g., 0, meaning no pain) to a high value (e.g., 7, meaning extreme pain). A patient experiencing pain is asked to choose a number between the low value and the high value to represent the degree of pain experienced. Likert scales can also be used, e.g., to measure fatigue. Likert scales and their use are described, e.g., in U.S. Pat. Nos. 6,623,040 and 6,766,319.

The Lequesne index and the Western Ontario and McMaster Universities (WOMAC) osteoarthritis index assess pain, function, and stiffness in the knee and hip of OA patients using self-administered questionnaires. Both knee and hip are encompassed by the WOMAC, whereas there is one Lequesne questionnaire for the knee and a separate one for the hip. These questionnaires are useful because they contain more information content in comparison with VAS or Likert. Both the WOMAC index and the Lequesne index questionnaires have been extensively validated in OA, including in surgical settings (e.g., knee and hip arthroplasty). Their metric characteristics do not differ significantly.

The AUSCAN (Australian-Canadian hand arthritis) index employs a valid, reliable, and responsive patient self-reported questionnaire. In one instance, this questionnaire contains 15 questions within three dimensions (Pain, 5 questions; Stiffness, 1 question; and Physical function, 9 questions). An AUSCAN index may utilize, e.g., a Likert or a VAS scale.

The Piper Fatigue scale is a 41-item measure of fatigue developed for research purposes and tested with oncology patients (Piper et al. (1989), The development of an instrument to measure the subjective dimension of fatigue. In S. Funk, E. Tornquist, M. Champagne, & R. Wiese (Eds.). Key aspects of comfort; Management of pain, fatigue, and nausea (pp. 199-207). New York: Springer.) The Multidimensional Assessment of Fatigue (MAF) scale, a revision of the Piper Fatigue scale, contains 15 items and measures four dimensions of fatigue: severity (#1-2), distress (#3), degree of interference in activities of daily living (#4-14), and frequency (#15), with scores ranging from 1 (no fatigue) to 50 (severe fatigue). The MAF has been validated in RA patients (Belza, J. Rheumatol. 22:639-643, 1995).

### EXAMPLES

The following examples are meant to illustrate the invention and are not meant to limit the invention in any way.

### Example 1

A multi-center, randomized, blinded, placebo-controlled 42-day study was conducted to test the effects of a novel syncretic drug containing 3 mg prednisolone and 200-400 mg dipyridamole. Patients with hand osteoarthritis were enrolled into the study. In order to be eligible, patients had to have more than one swollen and tender joint, a Kellgren-Lawrence (K-L) score of 2 or more on radiographs, and a score of at least 30 mm pain on the 100 mm AUSCAN (Australian-Canadian) visual analog scale. The primary endpoint was a reduction in pain using the AUSCAN pain subscale index at Day 42. Eighty-three patients were enrolled at four centers in Norway and randomized equally between the two treatment groups. Ninety-three percent were female, and the mean age was 60 years. Eleven patients (13%) had a K-L score of 2, with the remaining 72 patients (87%) having a score of 3 or more. At Day 42, there was a statistically significant reduction (p=0.006, where the p value is determined using ANCOVA adjusted for the baseline value, testing the null hypothesis that there is no difference in mean AUSCAN pain change between the treatment groups at Day 42) from baseline in the AUSCAN pain score in the group receiving the dipyridamole/prednisolone combination in comparison to the placebo group, as shown in Table 2. The mean change from baseline in the group receiving the dipyridamole/prednisolone combination was −102.4 mm, and the mean change from baseline in the placebo group was −30.9 mm. The difference between the adjusted means was 71.5 mm (95% CI: 16.05, 126.87).

TABLE 2
Summary of AUSCAN Pain Score Data

| | Placebo (N = 33) | | Dipyridamole/Prednisolone (N = 26) | |
|---|---|---|---|---|
| | | Change from Baseline | | Change from Baseline |
| Visit | Joint Pain (mm) | in Joint Pain (mm) | Joint Pain (mm) | in Joint Pain (mm) |

Baseline

| | | |
|---|---|---|
| N | 33 | 26 |
| Mean (Std Dev) | 319.0 (85.97) | 309.3 (83.21) |
| Median | 342.0 | 304.5 |
| Min, Max | 72, 460 | 58, 455 |

EOS/Day 42

| | | | | |
|---|---|---|---|---|
| N | 33 | 33 | 26 | 26 |
| LS mean (SE) | | −30.9 (18.34) | | −102.4 (20.67) |
| Mean (Std Dev) | 286.3 (111.91) | −32.7 (101.59) | 209.2 (120.01) | −100.1 (120.22) |
| Median | 298.0 | −8.0 | 215.0 | −63.0 |
| Min, Max | 70, 484 | −297, 143 | 14, 389 | −324, 74 |
| one-sided p-value (a) | | | | 0.0062 |
| Difference (95% CI) | | | | 71.5 (16.05, 126.87) |

a) P-value from ANCOVA adjusted for baseline value testing the null hypothesis there is no difference in mean AUSCAN pain change between the treatment groups at EOS/Day 42. (one-sided result for difference of LS means is presented.)
Note:
Pain Score is the sum of items 1-5 on the AUSCAN Hand Osteoarthritis Index.

These data are also shown in Tables 3 (for patients who complied with the study protocol) and Table 4 (for all enrolled patients), along with additional clinical measures, including physical function, stiffness, joint pain, and patient global assessment score (in which patients were asked to evaluate their osteoarthritis taking all symptoms into consideration). Tables 3 and 4 show the baseline mean (SD), changes at Day 42 in comparison to baseline (LS mean (SEM)), and treatment effect (mean difference (95% CI) study combination minus placebo). Percentage improvements over baseline (100×mean improvement/mean baseline) for per-protocol patients were 33% (pain), 20% (physical function), 32% (stiffness), 39% (joint pain), and 38% (patient global assessment).

TABLE 3
Summary of Efficacy Variable Data
(Per-Protocol Population)

| Baseline | | | | | |
|---|---|---|---|---|---|
| Dipyridamole/ | | Changes | | Treatment Effect | |
| Prednisolone | Placebo | Dipyridamole/ | | | p- |
| (N = 26) | (N = 33) | Prednisolone | Placebo | Difference | value |

AUSCAN

| | | | | | | |
|---|---|---|---|---|---|---|
| Pain (mm) | 309.3 | 319.0 | −102.4 | −30.9 | 71.5 | 0.006 |
| | (83.2) | (86.0) | (20.7) | (18.3) | (16.1, 126.9) | |
| Physical | 584.2 | 638.5 | −115.8 | −53.1 | 62.8 | 0.08 |
| function | (170.2) | (140.2) | (32.8) | (29.1) | (−25.8, 151.3) | |
| (mm) | | | | | | |
| Stiffness | 62.9 | 67.8 | −20.3 | −8.3 | 12.0 | 0.02 |
| (mm) | (17.4) | (19.8) | (4.4) | (3.9) | (0.2, 23.9) | |

VAS

| | | | | | | |
|---|---|---|---|---|---|---|
| Joint Pain | 59.8 | 62.9 | −23.5 | −6.3 | 17.2 | 0.002 |
| (mm) | (19.5) | (16.7) | (4.4) | (3.9) | (5.5, 28.9) | |
| Patient | 61.5 | 62.5 | −23.4 | −4.6 | 18.8 | <0.001 |
| Global (mm) | (17.5) | (17.6) | (4.0) | (3.6) | (8.1, 29.5) | |

TABLE 4
Summary of Efficacy Variable Data
(Intent-to-Treat Population)

| Baseline | | | | | |
|---|---|---|---|---|---|
| Dipyridamole/ | | Changes | | Treatment Effect | |
| Prednisolone | Placebo | Dipyridamole/ | | | p- |
| (N = 42) | (N = 41) | Prednisolone | Placebo | Difference | value |

AUSCAN

| | | | | | | |
|---|---|---|---|---|---|---|
| Pain (mm) | 289.4 | 304.5 | −70.9 | −20.2 | 50.8 | 0.01 |
| | (101.2) | (96.8) | (15.1) | (15.2) | (8.1, 93.5) | |
| Physical | 561.3 | 610.3 | −73.2 | −32.6 | 40.5 | 0.12 |
| function | (175.4) | (157.9) | (24.2) | (24.5) | (−28.4, 109.5) | |
| (mm) | | | | | | |
| Stiffness | 61.1 | 64.5 | −15.2 | −7.7 | 7.5 | 0.05 |
| (mm) | (18.0) | (21.2) | (3.2) | (3.3) | (−1.7, 16.7) | |

VAS

| | | | | | | |
|---|---|---|---|---|---|---|
| Joint Pain | 58.3 | 62.1 | −18.6 | −6.3 | 12.3 | 0.005 |
| (mm) | (20.1) | (16.9) | (3.3) | (3.3) | (3.0, 21.5) | |
| Patient | 58.0 | 62.3 | −15.9 | −4.2 | 11.7 | 0.007 |
| Global (mm) | (19.5) | (17.9) | (3.2) | (3.3) | (2.5, 20.8) | |

For each patient, the number of hand joints exhibiting joint tenderness/pain on joint motion, impairment in mobility, soft tissue swelling, or bony swelling was periodically counted and recorded during the study to determine the mean number of affected joints for each category. The findings are shown in Tables 5, 7, 9, and 11 (for patients who complied with the study protocol) and Tables 6, 8, 10, and 12 (for all enrolled patients).

TABLE 5
Joint Count (Tenderness/Pain on Joint Motion) by Study Visit
(Per-Protocol Population)

| | Placebo (N = 33) | | Dipyridamole/Prednisolone (N = 26) | |
|---|---|---|---|---|
| Visit | Number of Joints | Change from Baseline | Number of Joints | Change from Baseline |

Baseline

| | | |
|---|---|---|
| N | 33 | 26 |
| Mean (Std Dev) | 11.4 (5.30) | 11.2 (4.89) |
| Median | 12.0 | 11.0 |
| Min, Max | 2, 22 | 3, 20 |

Day 7

| | | | | |
|---|---|---|---|---|
| N | 33 | 33 | 26 | 26 |
| Mean (Std Dev) | 9.5 (5.94) | −1.8 (4.42) | 9.0 (5.44) | −2.1 (3.68) |
| Median | 10.0 | −1.0 | 9.0 | −1.0 |
| Min, Max | 0, 22 | −15, 7 | 0, 20 | −14, 2 |

Day 14

| | | | | |
|---|---|---|---|---|
| N | 33 | 33 | 26 | 26 |
| Mean (Std Dev) | 8.5 (5.86) | −2.9 (5.11) | 7.6 (6.25) | −3.5 (5.36) |
| Median | 9.0 | −2.0 | 5.5 | −2.0 |
| Min, Max | 0, 22 | −16, 5 | 0, 20 | −18, 6 |

Day 28

| | | | | |
|---|---|---|---|---|
| N | 33 | 33 | 26 | 26 |
| Mean (Std Dev) | 8.0 (5.34) | −3.3 (5.21) | 5.4 (4.90) | −5.7 (6.20) |
| Median | 7.0 | −3.0 | 5.0 | −4.5 |
| Min, Max | 0, 22 | −16, 8 | 0, 15 | −19, 2 |

EOS/Day 42

| | | | | |
|---|---|---|---|---|
| N | 33 | 33 | 26 | 26 |
| LS mean (SE) | | −2.8 (1.00) | | −5.5 (1.13) |
| Mean (Std Dev) | 8.5 (6.49) | −2.9 (6.08) | 5.8 (5.45) | −5.4 (6.92) |
| Median | 8.0 | −1.0 | 3.5 | −5.5 |
| Min, Max | 0, 22 | −18, 7 | 0, 16 | −18, 4 |
| one-sided p-value (a) | | | | 0.0431 |
| Difference (95% CI) | | | | 2.6 (−0.39, 5.65) |

TABLE 6
Joint Count (Tenderness/Pain on Joint Motion) by Study Visit
(Intent-to-Treat Population)

| | Placebo (N = 41) | | Dipyridamole/Prednisolone (N = 42) | |
|---|---|---|---|---|
| Visit | Number of Joints | Change from Baseline | Number of Joints | Change from Baseline |

Baseline

| | | |
|---|---|---|
| N | 41 | 42 |
| Mean (Std Dev) | 11.0 (5.15) | 11.2 (4.95) |
| Median | 10.0 | 11.0 |
| Min, Max | 2, 22 | 3, 22 |

Day 7

| | | | | |
|---|---|---|---|---|
| N | 40 | 40 | 37 | 37 |
| Mean (Std Dev) | 9.5 (5.64) | −1.6 (4.26) | 9.4 (5.31) | −2.1 (3.71) |
| Median | 9.0 | −1.0 | 9.0 | −1.0 |
| Min, Max | 0, 22 | −15, 7 | 0, 22 | −14, 2 |

Day 14

| | | | | |
|---|---|---|---|---|
| N | 39 | 39 | 27 | 27 |
| Mean (Std Dev) | 8.7 (5.69) | −2.5 (4.99) | 7.4 (6.22) | −3.6 (5.28) |
| Median | 9.0 | −1.0 | 5.0 | −2.0 |
| Min, Max | 0, 22 | −16, 6 | 0, 20 | −18, 6 |

Day 28

| | | | | |
|---|---|---|---|---|
| N | 35 | 35 | 26 | 26 |
| Mean (Std Dev) | 8.1 (5.23) | −3.1 (5.14) | 5.4 (4.90) | −5.7 (6.20) |
| Median | 7.0 | −2.0 | 5.0 | −4.5 |
| Min, Max | 0, 22 | −16, 8 | 0, 15 | −19, 2 |

EOS/Day 42

| | | | | |
|---|---|---|---|---|
| N | 41 | 41 | 41 | 41 |
| LS mean (SE) | | −2.6 (0.83) | | −4.1 (0.83) |
| Mean (Std Dev) | 8.4 (6.16) | −2.6 (5.73) | 7.1 (5.44) | −4.2 (6.08) |
| Median | 8.0 | −1.0 | 6.0 | −3.0 |
| Min, Max | 0, 22 | −18, 7 | 0, 22 | −18, 4 |
| one-sided p-value (a) | | | | 0.1074 |
| Difference (95% CI) | | | | 1.5 (−0.87, 3.80) |

TABLE 7
Joint Count (Limited Mobility) by Study Visit
(Per-Protocol Population)

| | Placebo (N = 33) | | Dipyridamole/Prednisolone (N = 26) | |
|---|---|---|---|---|
| Visit | Number of Joints | Change from Baseline | Number of Joints | Change from Baseline |

Baseline

| | | |
|---|---|---|
| N | 33 | 26 |
| Mean (Std Dev) | 8.3 (5.41) | 6.7 (6.29) |
| Median | 9.0 | 4.5 |
| Min, Max | 0, 21 | 0, 21 |

Day 7

| | | | | |
|---|---|---|---|---|
| N | 33 | 33 | 26 | 26 |
| Mean (Std Dev) | 6.9 (5.63) | −1.4 (3.01) | 6.6 (6.19) | −0.1 (1.57) |
| Median | 6.0 | 0.0 | 4.5 | 0.0 |
| Min, Max | 0, 22 | −13, 2 | 0, 20 | −3, 4 |

Day 14

| | | | | |
|---|---|---|---|---|
| N | 33 | 33 | 26 | 26 |
| Mean (Std Dev) | 7.4 (6.00) | −0.9 (3.82) | 5.5 (5.52) | −1.2 (4.12) |
| Median | 6.0 | 0.0 | 4.0 | 0.0 |
| Min, Max | 0, 22 | −13, 5 | 0, 22 | −14, 5 |

Day 28

| | | | | |
|---|---|---|---|---|
| N | 33 | 33 | 26 | 26 |
| Mean (Std Dev) | 6.5 (5.32) | −1.8 (4.98) | 4.5 (4.72) | −2.2 (5.21) |
| Median | 6.0 | 0.0 | 2.0 | −1.0 |
| Min, Max | 0, 22 | −15, 5 | 0, 18 | −17, 6 |

EOS/Day 42

| | | | | |
|---|---|---|---|---|
| N | 33 | 33 | 26 | 26 |
| LS mean (SE) | | −1.2 (0.71) | | −2.2 (0.80) |
| Mean (Std Dev) | 6.8 (5.52) | −1.5 (3.64) | 4.8 (5.04) | −1.9 (5.67) |
| Median | 6.0 | −1.0 | 2.0 | −0.5 |
| Min, Max | 0, 22 | −10, 4 | 0, 18 | −17, 10 |
| one-sided p-value (a) | | | | 0.1744 |
| Difference (95% CI) | | | | 1.0 (−1.14, 3.16) |

TABLE 8
Joint Count (Limited Mobility) by Study Visit
(Intent-to-Treat Population)

| | Placebo (N = 41) | | Dipyridamole/Prednisolone (N = 42) | |
|---|---|---|---|---|
| Visit | Number of Joints | Change from Baseline | Number of Joints | Change from Baseline |

Baseline

| | | |
|---|---|---|
| N | 41 | 42 |
| Mean (Std Dev) | 8.5 (5.17) | 7.5 (6.34) |
| Median | 9.0 | 6.0 |
| Min, Max | 0, 21 | 0, 22 |

Day 7

| | | | | |
|---|---|---|---|---|
| N | 40 | 40 | 37 | 37 |
| Mean (Std Dev) | 7.0 (5.55) | −1.4 (2.91) | 7.9 (6.68) | 0.3 (3.05) |
| Median | 6.0 | 0.0 | 7.0 | 0.0 |
| Min, Max | 0, 22 | −13, 3 | 0, 22 | −7, 13 |

Day 14

| | | | | |
|---|---|---|---|---|
| N | 39 | 39 | 27 | 27 |
| Mean (Std Dev) | 8.1 (6.10) | −0.5 (3.80) | 5.5 (5.41) | −1.3 (4.11) |
| Median | 8.0 | 0.0 | 4.0 | 0.0 |
| Min, Max | 0, 22 | −13, 7 | 0, 22 | −14, 5 |

Day 28

| | | | | |
|---|---|---|---|---|
| N | 35 | 35 | 26 | 26 |
| Mean (Std Dev) | 6.5 (5.20) | −1.9 (4.93) | 4.5 (4.72) | −2.2 (5.21) |
| Median | 6.0 | 0.0 | 2.0 | −1.0 |
| Min, Max | 0, 22 | −15, 5 | 0, 18 | −17, 6 |

EOS/Day 42

| | | | | |
|---|---|---|---|---|
| N | 41 | 41 | 41 | 41 |
| LS mean (SE) | | −1.6 (0.61) | | −2.1 (0.61) |
| Mean (Std Dev) | 6.7 (5.43) | −1.8 (3.73) | 5.6 (4.81) | −1.9 (5.24) |
| Median | 6.0 | −1.0 | 5.0 | 0.0 |
| Min, Max | 0, 22 | −10, 4 | 0, 18 | −17, 10 |
| one-sided p-value (a) | | | | 0.2696 |
| Difference (95% CI) | | | | 0.5 (−1.18, 2.24) |

TABLE 9
Joint Count (Soft Tissue Swelling) by Study Visit
(Per-Protocol Population)

| | Placebo (N = 33) | | Dipyridamole/Prednisolone (N = 26) | |
|---|---|---|---|---|
| Visit | Number of Joints | Change from Baseline | Number of Joints | Change from Baseline |

Baseline

| | | |
|---|---|---|
| N | 33 | 26 |
| Mean (Std Dev) | 5.3 (4.48) | 6.3 (4.67) |
| Median | 4.0 | 4.0 |
| Min, Max | 0, 15 | 1, 18 |

Day 7

| | | | | |
|---|---|---|---|---|
| N | 33 | 33 | 26 | 26 |
| Mean (Std Dev) | 5.0 (4.56) | −0.3 (2.65) | 5.0 (5.27) | −1.2 (3.17) |
| Median | 3.0 | 0.0 | 3.5 | −1.0 |
| Min, Max | 0, 16 | −8, 5 | 0, 18 | −11, 4 |

Day 14

| | | | | |
|---|---|---|---|---|
| N | 33 | 33 | 26 | 26 |
| Mean (Std Dev) | 4.5 (4.35) | −0.8 (3.25) | 3.7 (4.60) | −2.6 (4.35) |
| Median | 3.0 | 0.0 | 2.0 | −1.5 |
| Min, Max | 0, 15 | −9, 6 | 0, 16 | −16, 5 |

Day 28

| | | | | |
|---|---|---|---|---|
| N | 33 | 33 | 26 | 26 |
| Mean (Std Dev) | 3.6 (4.20) | −1.7 (4.75) | 3.0 (4.01) | −3.3 (4.56) |
| Median | 2.0 | 0.0 | 2.0 | −2.0 |
| Min, Max | 0, 18 | −15, 7 | 0, 16 | −18, 2 |

EOS/Day 42

| | | | | |
|---|---|---|---|---|
| N | 33 | 33 | 26 | 26 |
| LS mean (SE) | | −1.9 (0.56) | | −3.4 (0.63) |
| Mean (Std Dev) | 3.7 (3.69) | −1.6 (4.01) | 2.5 (3.61) | −3.8 (4.38) |
| Median | 3.0 | 0.0 | 1.5 | −2.0 |
| Min, Max | 0, 14 | −10, 6 | 0, 13 | −18, 0 |
| one-sided p-value (a) | | | | 0.0349 |
| Difference (95% CI) | | | | 1.6 (−0.13, 3.24) |

TABLE 10
Joint Count (Soft Tissue Swelling) by Study Visit
(Intent-to-Treat Population)

| | Placebo (N = 41) | | Dipyridamole/Prednisolone (N = 42) | |
|---|---|---|---|---|
| Visit | Number of Joints | Change from Baseline | Number of Joints | Change from Baseline |

Baseline

| | | |
|---|---|---|
| N | 41 | 42 |
| Mean (Std Dev) | 5.4 (4.59) | 6.1 (4.87) |
| Median | 3.0 | 4.0 |
| Min, Max | 0, 16 | 0, 18 |

Day 7

| | | | | |
|---|---|---|---|---|
| N | 40 | 40 | 37 | 37 |
| Mean (Std Dev) | 5.0 (4.55) | −0.5 (2.65) | 5.1 (5.34) | −1.3 (3.27) |
| Median | 3.0 | 0.0 | 3.0 | −1.0 |
| Min, Max | 0, 16 | −8, 5 | 0, 18 | −11, 4 |

Day 14

| | | | | |
|---|---|---|---|---|
| N | 39 | 39 | 27 | 27 |
| Mean (Std Dev) | 4.6 (4.43) | −0.8 (3.10) | 3.6 (4.52) | −3.0 (4.71) |
| Median | 3.0 | 0.0 | 2.0 | −2.0 |
| Min, Max | 0, 15 | −9, 6 | 0, 16 | −16, 5 |

Day 28

| | | | | |
|---|---|---|---|---|
| N | 35 | 35 | 26 | 26 |
| Mean (Std Dev) | 3.8 (4.34) | −1.7 (4.64) | 3.0 (4.01) | −3.3 (4.56) |
| Median | 2.0 | 0.0 | 2.0 | −2.0 |
| Min, Max | 0, 18 | −15, 7 | 0, 16 | −18, 2 |

EOS/Day 42

| | | | | |
|---|---|---|---|---|
| N | 41 | 41 | 41 | 41 |
| LS mean (SE) | | −1.6 (0.54) | | −2.6 (0.54) |
| Mean (Std Dev) | 3.9 (3.89) | −1.4 (3.77) | 3.4 (4.75) | −2.7 (4.19) |
| Median | 3.0 | 0.0 | 2.0 | −2.0 |
| Min, Max | 0, 14 | −10, 6 | 0, 18 | −18, 5 |
| one-sided p-value (a) | | | | 0.1065 |
| Difference (95% CI) | | | | 1.0 (−0.56, 2.47) |

TABLE 11
Joint Count (Bony Swelling) by Study Visit
(Per-Protocol Population)

| | Placebo (N = 33) | | Dipyridamole/Prednisolone (N = 26) | |
|---|---|---|---|---|
| Visit | Number of Joints | Change from Baseline | Number of Joints | Change from Baseline |

Baseline

| | | |
|---|---|---|
| N | 33 | 26 |
| Mean (Std Dev) | 14.0 (4.31) | 14.9 (4.57) |
| Median | 15.0 | 13.5 |
| Min, Max | 5, 22 | 7, 22 |

Day 7

| | | | | |
|---|---|---|---|---|
| N | 33 | 33 | 26 | 26 |
| Mean (Std Dev) | 14.0 (4.19) | 0.0 (1.21) | 15.1 (4.62) | 0.2 (1.75) |
| Median | 14.0 | 0.0 | 16.0 | 0.0 |
| Min, Max | 5, 20 | −3, 4 | 7, 21 | −3, 5 |

Day 14

| | | | | |
|---|---|---|---|---|
| N | 33 | 33 | 26 | 26 |
| Mean (Std Dev) | 14.5 (4.51) | 0.5 (1.58) | 14.8 (4.83) | −0.1 (1.83) |
| Median | 17.0 | 0.0 | 15.5 | 0.0 |
| Min, Max | 5, 20 | −3, 5 | 7, 22 | −5, 4 |

Day 28

| | | | | |
|---|---|---|---|---|
| N | 33 | 33 | 26 | 26 |
| Mean (Std Dev) | 14.2 (4.65) | 0.3 (1.99) | 14.8 (4.93) | −0.1 (2.34) |
| Median | 16.0 | 0.0 | 14.5 | 0.0 |
| Min, Max | 5, 21 | −5, 5 | 7, 22 | −6, 7 |

EOS/Day 42

| | | | | |
|---|---|---|---|---|
| N | 33 | 33 | 26 | 26 |
| LS mean (SE) | | 0.2 (0.38) | | 0.5 (0.43) |
| Mean (Std Dev) | 14.2 (4.89) | 0.2 (1.98) | 15.3 (4.94) | 0.5 (2.39) |
| Median | 15.0 | 0.0 | 16.0 | 0.0 |
| Min, Max | 5, 22 | −3, 8 | 6, 22 | −3, 8 |
| one-sided p-value (a) | | | | 0.6881 |
| Difference (95% CI) | | | | −0.3 (−1.44, 0.87) |

TABLE 12
Joint Count (Bony Swelling) by Study Visit
(Intent-to-Treat Population)

| | Placebo (N = 41) | | Dipyridamole/Prednisolone (N = 42) | |
|---|---|---|---|---|
| Visit | Number of Joints | Change from Baseline | Number of Joints | Change from Baseline |

Baseline

| | | |
|---|---|---|
| N | 41 | 42 |
| Mean (Std Dev) | 13.6 (4.23) | 15.3 (4.40) |
| Median | 14.0 | 15.0 |
| Min, Max | 5, 22 | 7, 22 |

Day 7

| | | | | |
|---|---|---|---|---|
| N | 40 | 40 | 37 | 37 |
| Mean (Std Dev) | 13.7 (4.03) | 0.0 (1.23) | 15.3 (4.27) | 0.3 (1.84) |
| Median | 14.0 | 0.0 | 16.0 | 0.0 |
| Min, Max | 5, 20 | −3, 4 | 7, 21 | −4, 5 |

Day 14

| | | | | |
|---|---|---|---|---|
| N | 39 | 39 | 27 | 27 |
| Mean (Std Dev) | 13.9 (4.43) | 0.1 (2.05) | 14.8 (4.74) | −0.3 (2.03) |
| Median | 14.0 | 0.0 | 15.0 | 0.0 |
| Min, Max | 5, 20 | −8, 5 | 7, 22 | −5, 4 |

Day 28

| | | | | |
|---|---|---|---|---|
| N | 35 | 35 | 26 | 26 |
| Mean (Std Dev) | 14.1 (4.54) | 0.1 (2.06) | 14.8 (4.93) | −0.1 (2.34) |
| Median | 14.0 | 0.0 | 14.5 | 0.0 |
| Min, Max | 5, 21 | −5, 5 | 7, 22 | −6, 7 |

EOS/Day 42

| | | | | |
|---|---|---|---|---|
| N | 41 | 41 | 41 | 41 |
| LS mean (SE) | | 0.1 (0.32) | | 0.4 (0.32) |
| Mean (Std Dev) | 13.7 (4.86) | 0.1 (1.92) | 15.7 (4.53) | 0.4 (2.10) |
| Median | 14.0 | 0.0 | 16.0 | 0.0 |
| Min, Max | 5, 22 | −3, 8 | 6, 22 | −3, 8 |
| one-sided p-value (a) | | | | 0.7793 |
| Difference (95% CI) | | | | −0.4 (−1.26, 0.55) |

It can be concluded from the study that the dipyridamole/prednisolone combination tested in the study demonstrated efficacy in reducing pain, tenderness, impairment in mobility, and swelling in hand osteoarthritis, and that it was generally well tolerated.

The study described herein was conducted according to the following guidelines.

The study drug combination, or placebo, was administered orally twice daily, once at 8 AM and once at 1 PM. Tablets for the study drug combination were blister packed for two dose levels and administered as shown in Tables 13 and 14; placebo tablets were administered as shown in Table 15.

TABLE 13
Study Drug Combination, Days 1-7

| 8 A.M. | 8 A.M. | 1 P.M. |
|---|---|---|
| 100 mg dipyridamole | 1 mg prednisolone | 1 mg prednisolone |
| placebo (blue) | 1 mg prednisolone | 100 mg dipyridamole |

TABLE 14
Study Drug Combination, Days 8-42

| 8 A.M. | 8 A.M. | 1 P.M. |
|---|---|---|
| 100 mg dipyridamole | 1 mg prednisolone | 1 mg prednisolone |
| 100 mg dipyridamole | 1 mg prednisolone | 100 mg dipyridamole |
| | | 100 mg dipyridamole |

TABLE 15
Placebo, Days 1-42

| 8 A.M. | 8 A.M. | 1 P.M. |
|---|---|---|
| placebo (blue) | placebo (white) | placebo (white) |
| placebo (blue) | placebo (white) | placebo (blue) |
| | | placebo (blue) |

For each patient enrolled, a complete medical history was obtained at the initial Screening visit for each patient. The medical history included demographic background information and history of osteoarthritis.

A complete physical examination was conducted at the Screening visit. In addition, a complete physical examination was conducted at the End of Study and Follow-up visits to evaluate any changes from baseline status. This included joint examination for tenderness and swelling.

Vital signs, including heart rate, respiratory rate, blood pressure, and body temperature, were measured at each visit. Height and weight were measured during the Screening visit.

The AUSCAN (visual analog scale (VAS)) test, a valid, reliable, and responsive tri-dimensional patient self-reported questionnaire containing 15 questions within three dimensions (Pain, 5 questions; Stiffness, 1 question; and Physical function, 9 questions), was performed and recorded at each visit, with the exception of the Follow-up visit.

The number of affected joints of the hand was counted and recorded at each visit, with the exception of the Follow-up visit. This included joint examination for tenderness, limited mobility, soft tissue swelling, and bony swelling. The joints used in the analysis were the distal interphalangeal, proximal interphalangeal, interphalangeal, metacarpophalangeal, and carpometacarpal joints (total of 22 for both hands).

Joint pain of the hand was rated on a standard visual analogue scale (VAS). The scale consists of a 10 cm (100 mm) horizontal line with the phrases “no pain” and “the worst pain you could possibly imagine” placed at the left and right ends, respectively. Patients were instructed to complete the visual analogue pain scale by marking the spot on the line correlating to the level of pain experienced. The level of pain was calculated by measuring (in millimeters) the distance from the left end of the scale to the mark. Joint pain of the hand (VAS) assessment was performed and recorded at each visit, with the exception of the Follow-up visit.

The Patient Global (VAS) test was used to assess patient global pain by the 100 mm visual analogue scale. It was rated for all pain experienced at the time of the study visit. Patient Global (VAS) was performed and recorded at each visit, with the exception of the Follow-up visit.

An X-ray was obtained during the Screening visit if an X-ray had not been performed within six months before the Screening visit, or if the patient had not previously demonstrated OA abnormalities. If the patient had an X-ray performed within six months before the Screening visit, then that X-ray was used for screening purposes. The radiological severity of OA was assessed by the Kellgren-Lawrence score system.

Monitoring of adverse events (AE) was conducted throughout the study. New adverse events, including serious adverse events (SAEs), were captured on the Case Report Forms (CRFs) through the Follow-up visit. Adverse events were followed in accordance with good clinical practice. Serious adverse events were immediately reported and monitored until they were resolved or clearly determined to be due to a patient's stable or chronic condition or inter-current illness(es).

All concomitant medications, procedures, and supportive therapy were recorded at all visits.

Blood samples for a rheumatoid factor test were collected at the Screening visit. Blood samples for an erythrocyte sedimentation rate test were collected at the Screening visit and on Day 42.

Blood samples for analysis of hematocrit, hemoglobin, red blood cell (RBC) count, platelet count, and white blood cell (WBC) count with differential were collected at the Screening visit, Day 28, Day 42, and Day 56. All samples were collected in the morning. Patients were instructed to fast prior to sample collection.

Blood samples for analysis of sodium, potassium, chloride, magnesium, and calcium levels were collected at the Screening visit, Day 28, Day 42, and Day 56. All samples were collected in the morning. Patients were instructed to fast prior to sample collection.

Blood samples for analysis of blood urea nitrogen (BUN), alkaline phosphatase, serum creatinine, bilirubin (total), uric acid, AST (SGOT), lactate dehydrogenase (LDH), ALT (SGPT), albumin, and glucose were drawn at the Screening visit; Day 28, Day 42, and Day 56. All samples were collected in the morning. Patients were instructed to fast prior to sample collection.

A urine pregnancy test was performed for women of child-bearing potential at the Screening visit. It was required that the results be available and negative before dosing. If a woman became pregnant or suspected she was pregnant while participating in this study, she was required to inform her treating physician immediately and permanently discontinue the study drug.

Serum samples for analysis of CRP, TNFα, IL-1, IL-2, IL-6, IL-8, IL-12, and IFNγ, were taken at the Baseline visit, as well as Day 7, Day 14, Day 28, and Day 42. Samples were drawn after the patient had taken the morning study drug dose.

Patients were informed that they have the right to withdraw from the study at any time for any reason, without prejudice to their medical care. The investigator also had the right to withdraw patients from the study for any of the following reasons: inter-current illness; occurrence of an unacceptable adverse event; patient request; protocol violations; administrative reasons; failure to return for follow-up; or general or specific changes in the patient's condition unacceptable for further treatment in the judgment of the investigator.

At the time of withdrawal, all appropriate study procedures were completed. The primary reason for a patient's withdrawal from the study was recorded. If patients withdrew prior to randomization, they were replaced.

### Example 2

A multi-center, randomized, blinded, placebo-controlled 42-day study was conducted to compare the effect of dipyridamole/prednisolone plus DMARD therapy to placebo plus DMARD therapy on serum CRP and cytokines in subjects with RA. A total of 59 subjects diagnosed with moderate to severe RA were enrolled. A summary of subject demographics is shown in Table 16. To be eligible for study enrollment, subjects must have had a serum CRP level of at least 2.2 mg/L, a Disease Activity Score (DAS28) of 4.5 or greater, and must have been on DMARD therapy for at least three months and have been on a stable dose for at least 28 days at the time of screening.

TABLE 16
Subject Demographics for Dipyridamole/Prednisolone + DMARD
Study
(Intent-to-Treat Population)

| | | |
|---|---|---|
| | Dipyridamole/ | |
| Placebo + | Prednisolone + | |
| DMARD | DMARD | |
| (N = 32) | (N = 27) | Total (N = 59) |

Gender, n (%)

| | | | |
|---|---|---|---|
| Male | 8 (25) | 5 (19) | 13 (22) |
| Female | 24 (75) | 22 (81) | 46 (78) |

Race, n (%)

| | | | |
|---|---|---|---|
| Caucasian | 30 (94) | 27 (100) | 57 (97) |
| Black | 1 (3) | 0 | 1 (2) |
| Other | 1 (3) | 0 | 1 (2) |

Age (years)

| | | | |
|---|---|---|---|
| n | 32 | 27 | 59 |
| Mean (SD) | 59.1 (12.31) | 57.8 (7.54) | 58.5 (10.34) |
| Median | 59.5 | 56.0 | 59.0 |
| Min, Max | 31, 78 | 45, 76 | 31, 78 |

Height (cm)

| | | | |
|---|---|---|---|
| n | 32 | 27 | 59 |
| Mean (SD) | 164.10 (7.344) | 165.07 (8.468) | 164.54 (7.824) |
| Median | 162.15 | 165.90 | 165.00 |
| Min, Max | 149.0, 175.0 | 149.0, 181.6 | 149.0, 181.6 |

Weight (kg)

| | | | |
|---|---|---|---|
| n | 32 | 27 | 59 |
| Mean (SD) | 74.99 (12.144) | 79.32 (17.095) | 76.97 (14.648) |
| Median | 74.75 | 77.90 | 76.60 |
| Min, Max | 48.4, 102.3 | 47.0, 111.5 | 47.0, 111.5 |

Abbreviation:
SD = standard deviation

Subjects were randomized 1:1 to treatment with either the dipyridamole/prednisolone combination or placebo. All eligible subjects received DMARD therapy in a standard dose. Study medication was administered orally twice a day at 8 AM and at 1 PM. Subjects received 3 mg prednisolone (2 mg in the AM, 1 mg in the PM) and 200 mg dipyridamole (100 mg in the AM, 100 mg in the PM) (or placebo equivalent) daily on Days 1 to 7 and then 3 mg prednisolone (2 mg in the AM, 1 mg in the PM) and 400 mg dipyridamole (200 mg in the AM, 200 mg in the PM) (or placebo equivalent) daily on Days 8 to 42. Results of the study for the per-protocol population (last observation carried forward) are summarized in Table 17.

TABLE 17
Summary of Treatment with Dipyridamole/Prednisolone + DMARD
and Placebo + DMARD on Efficacy Assessments from Baseline to
Day 42 of Study
(Per Protocol Population, Last Observation Carried Forward)

| | Placebo + DMARD | | Dipyridamole/Prednisolone + DMARD | |
|---|---|---|---|---|
| | (N = 27) | | (N = 19) | |
| | | Change from | | Change from |
| Assessment | Day 42 Value | Baseline^{a} | Day 42 Value | Baseline^{a} |

CRP (mg/L)

| | | | | |
|---|---|---|---|---|
| n | 27 | 27 | 19 | 19 |
| Mean (SD) | 15.9258 (18.85357) | 9.60 (74.154) | 9.4746 (16.03221) | −16.12 (94.985) |
| Median | 6.8410 | 18.90 | 5.0640 | −49.50 |
| Min, Max | 0.945, 84.921 | −98.6, 208.9 | 0.566, 70.337 | −93.4, 281.5 |
| 1-sided p-value^{b} | | | | 0.0235 |

ACR 20

| | | |
|---|---|---|
| n | 27 | 19 |
| Yes | 8 (30) | 12 (63) |
| No | 19 (70) | 7 (37) |
| P-value^{c} | | 0.02492 |

DAS 28
(scale 0 to 10)

| | | | | |
|---|---|---|---|---|
| n | 27 | 26 | 19 | 19 |
| LS mean (SE) | — | −0.7 (0.27) | — | −1.6 (0.32) |
| Mean (SD) | 6.466 (1.6812) | −0.697 (1.4351) | 5.395 (1.4497) | −1.521 (1.5738) |
| Median | 7.206 | −0.464 | 5.130 | −1.727 |
| Min, Max | 2.13, 8.66 | −3.72, 1.76 | 3.47, 8.47 | −4.38, 1.71 |
| 1-sided p-value^{d} | | | | 0.0163 |
| Difference | | | | 0.9 (0.08, 1.79) |
| (95% CI) | | | | |

HAQ_DI
(scale 0 to 3)

| | | | | |
|---|---|---|---|---|
| n | 27 | 27 | 19 | 19 |
| Mean (SD) | 1.662 (0.8448) | −9.487 (26.9652) | 1.362 (0.8679) | −24.316 (38.2286) |
| Median | 1.500 | 0.000 | 1.250 | −14.286 |
| Min, Max | 0.13, 3.00 | −83.3, 33.33 | 0.00, 3.00 | −100.00, 40.00 |
| 1-sided p-value^{b} | | | | 0.0386 |

Clinician's Global
Assessment (VAS)

| | | | | |
|---|---|---|---|---|
| n | 27 | 27 | 19 | 19 |
| Mean (SD) | 46.44 (24.377) | −9.92 (47.527) | 29.2 (18.33) | −35.88 (41.845) |
| Median | 52.00 | −6.00 | 21.0 | −35.50 |
| Min, Max | 4.0, 84.0 | −91.8, 62.5 | 6.0, 60.0 | −91.1, 50.0 |
| 1-sided | | | | 0.0320 |
| p-value^{b} | | | | |

Patient's Global
Assessment (VAS)

| | | | | |
|---|---|---|---|---|
| n | 27 | 27 | 19 | 19 |
| Mean (SD) | 44.52 (25.675) | 2.50 (75.449) | 34.6 (24.41) | −39.20 (35.532) |
| Median | 43.00 | 1.40 | 30.0 | −40.50 |
| Min, Max | 4.0, 95.0 | −91.5, 300.0 | 1.0, 88.0 | −97.1, 22.4 |
| 1-sided p-value^{b} | | | | 0.0102 |

Patient's Pain
Assessment (VAS)

| | | | | |
|---|---|---|---|---|
| n | 27 | 27 | 19 | 19 |
| Mean (SD) | 45.85 (28.417) | −16.12 (47.831) | 37.9 (23.77) | −43.67 (31.756) |
| Median | 46.00 | −14.30 | 36.0 | −41.40 |
| Min, Max | 4.0, 95.0 | −88.6, 108.1 | 1.0, 93.0 | −98.0, 10.7 |
| 1-sided p-value^{b} | | | | 0.0157 |

Fatigue (VAS)

| | | | | |
|---|---|---|---|---|
| n | 27 | 27 | 19 | 19 |
| LS mean (SE) | — | −14.3 (4.31) | — | −27.2 (5.14) |
| Mean (SD) | 45.1 (30.46) | −13.8 (24.11) | 34.0 (25.54) | −27.9 (22.58) |
| Median | 45.0 | −15.0 | 36.0 | −22.0 |
| Min, Max | 0.0, 84.0 | −60.0, 37.0 | 0.0, 83.0 | −74.0, −1.0 |
| 1-sided p-value^{d} | | | | 0.0314 |
| Difference | | | | 12.8 (−0.72, 26.41) |
| (95% CI) | | | | |

Erythrocyte
Sedimentation
Rate

| | | | | |
|---|---|---|---|---|
| n | 27 | 26 | 19 | 19 |
| Mean (SD) | 31.7 (16.88) | 4.54 (51.091) | 25.79 (17.687) | −26.89 (31.249) |
| Median | 27.0 | 3.15 | 23.00 | −32.70 |
| Min, Max | 6.0, 70.0 | −73.1, 188.2 | 6.0, 79.0 | −71.3, 62.5 |
| 1-sided p-value^{b} | | | | 0.0061 |

Number of
Tender Joints

| | | | | |
|---|---|---|---|---|
| n | 27 | 27 | 19 | 19 |
| Mean (SD) | 12.0 (7.99) | −18.93 (63.486) | 6.4 (6.26) | −44.89 (48.776) |
| Median | 14.0 | −16.70 | 4.0 | −50.00 |
| Min, Max | 0.0, 26.0 | −100.00, 160.0 | 0.0, 25.0 | −100.00, 85.7 |
| 1-sided p-value^{b} | | | | 0.0885 |

Number of
Swollen Joints

| | | | | |
|---|---|---|---|---|
| n | 27 | 27 | 19 | 19 |
| Mean (SD) | 10.1 (7.97) | −28.93 (54.755) | 6.2 (6.03) | 9.66 (188.732) |
| Median | 10.0 | −33.30 | 5.0 | −33.30 |
| Min, Max | 0.0, 23.0 | −100.00, 100.00 | 0.0, 21.0 | −100.00, 700.00 |
| 1-sided p-value^{b} | | | | 0.4073 |

^{a}For CRP, HAQ_DI, Clinician's and Patient's Global Assessments, Patient Pain Assessment, ESR, and Joint Counts, change from baseline was calculated for the percent change from baseline.
^{b}P-value from 1-sided Wilcoxon Rank Sum Test for percent reduction (of assessment) at Day 42.
^{c}P-value from 1-sided Fisher's Exact Test for ACR 20 response rate at Day 42.
^{d}P-value from 1-sided ANCOVA adjusted for baseline for mean DAS 28 or mean Fatigue VAS score at Day 42.
Abbreviations:
SE = standard error;
SD = standard deviation;
HAQ_DI = Health Assessment Questionnaire Disability Index.

Mean (±standard deviation) baseline values for CRP were not significantly different for placebo subjects (17.7253±17.12870 mg/L) and dipyridamole/prednisolone subjects (18.2552±21.39383 mg/L). The median CRP value at baseline for placebo (12.6650 mg/L) was slightly higher than the median value for dipyridamole/prednisolone subjects (9.1390 mg/L). For the primary endpoint (median percent change from baseline to Day 42), there was a statistically significant difference between dipyridamole/prednisolone subjects (49.50) and placebo subjects (−18.90) (p=0.0235; 1-sided Wilcoxon Rank Sum Test). A total of six (32%) of 19 dipyridamole/prednisolone subjects recorded at least a 70% reduction in CRP at Day 42, versus 1 (4%) of 27 placebo subjects (p=0.01463; 1-sided Fisher's Exact Test).

In addition, results from secondary and ancillary efficacy endpoints favored dipyridamole/prednisolone over placebo:
Twelve (63%) dipyridamole/prednisolone subjects had at least a 20% reduction in ACR 20 compared to eight (30%) placebo subjects (p=0.02492; 1-sided Fisher's Exact Test). For ACR 50 and ACR 70, there tended to be more dipyridamole/prednisolone subjects than placebo subjects who met the criteria.
The difference in the adjusted mean change from baseline (LS mean±SE) to Day 42 for the DAS 28 (scale 0 to 10) was statistically significant for dipyridamole/prednisolone subjects (1.6±0.32) versus placebo subjects (−0.7±0.27) (p=0.0163; 1-sided ANCOVA).
The median percent change from baseline to Day 42 results for the HAQ_DI (scale 0 to 3) was statistically significant in favor of dipyridamole/prednisolone subjects (−14.286) over placebo subjects (0.0) (p=0.0386; 1 sided Wicoxon Rank Sum Test).
The median percent change from baseline to Day 42 results for the Clinician's Global Assessment (VAS 0 to 100 mm) was statistically significant in favor of dipyridamole/prednisolone subjects (35.50) over placebo subjects (−6.00) (p=0.0320; 1 sided Wicoxon Rank Sum Test).
The median percent change from baseline to Day 42 results for the Patient's Global Assessment (VAS 0 to 100 mm) was statistically significant in favor of dipyridamole/prednisolone subjects (40.50) over placebo subjects (1.40) (p=0.0102; 1 sided Wicoxon Rank Sum Test).
The median percent change from baseline to Day 42 results for the Patient Pain Assessment (VAS 0 to 100 mm) was statistically significant in favor of dipyridamole/prednisolone subjects (41.40) over placebo subjects (−14.30) (p==0.0157; 1 sided Wicoxon Rank Sum Test).
The difference in the adjusted mean change from baseline (LS mean±SE) to Day 42 for the Fatigue VAS (0 to 100 mm) was statistically significant for dipyridamole/prednisolone subjects (27.2±5.14) versus placebo subjects (−14.3±4.31) (p=0.0314; 1-sided ANCOVA).
The median percent change from baseline to Day 42 results for the ESR was statistically significant in favor of dipyridamole/prednisolone subjects (32.70) over placebo subjects (3.15) (p=0.0061; 1 sided Wicoxon Rank Sum Test).
A trend was observed in favor of dipyridamole/prednisolone subjects versus placebo subjects for the overall number of tender joints (baseline through Day 42). The numbers of swollen joints was not clinically remarkable between the two treatments.

In general, results for the Intent-to-Treat Population (last observation carried forward) were not remarkably different than those for the Per Protocol Population (last observation carried forward).

It can be concluded from the study that the dipyridamole/prednisolone plus DMARD therapy tested in the study demonstrated efficacy in lowering CRP levels in subjects with RA, and also demonstrated efficacy in the secondary and ancillary efficacy measures tested in the study.

### Example 3

The study described in Example 2 was extended to include the collection of fatigue information. Fatigue was measured by two separate instruments: a single-question fatigue VAS, and a composite measure, the Multidimensional Assessment of Fatigue (MAF) scale.

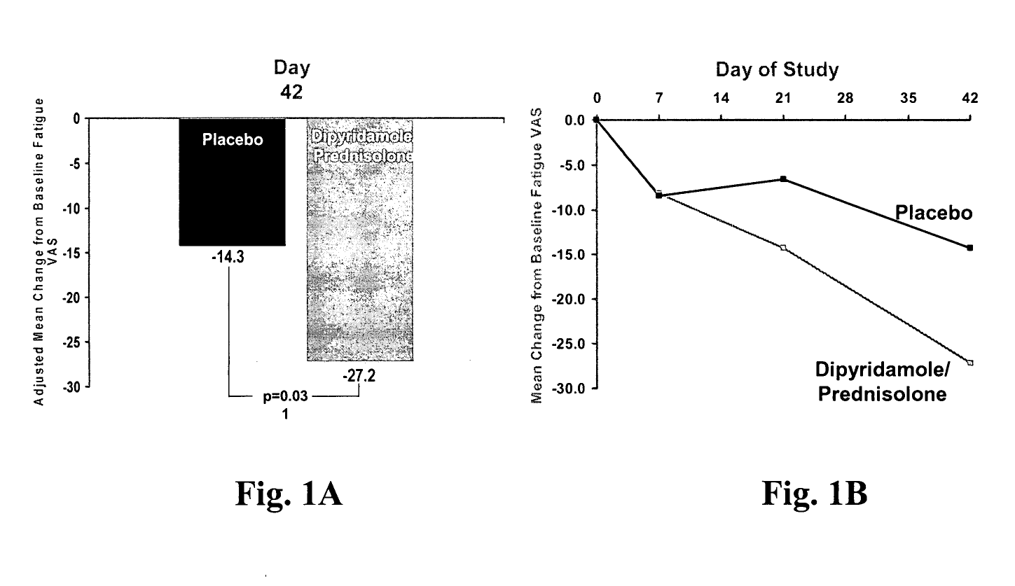

For the VAS measure, patients were asked, “How fatigued (tired) have you felt in the last week?” At Day 42, there was a statistically significant reduction (p=0.031) from baseline in the VAS fatigue score in the group receiving the dipyridamole/prednisolone combination+DMARD therapy in comparison to the placebo+DMARD therapy group, as shown in FIG. 1A. VAS measurements taken over the course of the 42-day study for the placebo group and the combination group are shown in FIG. 1B. Mean baseline VAS values were 58.9 mm for placebo and 61.9 mm for the dipyridamole/prednisolone combination.

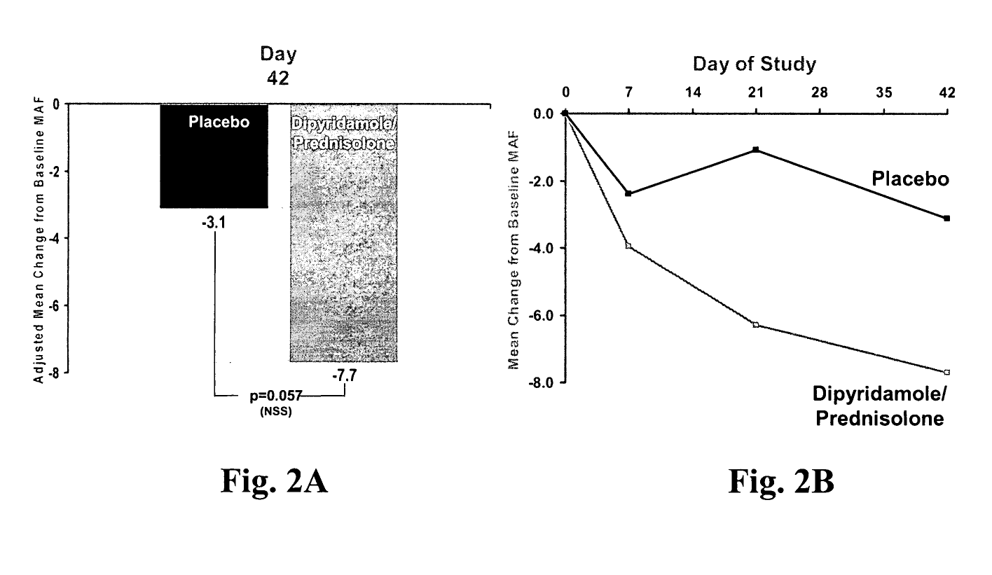

For the MAF measure, patients were asked to reflect on fatigue patterns for the past week and answer fifteen questions measuring four dimensions of fatigue: severity, distress, degree of interference in activities of daily living, and frequency. At Day 42, there was a reduction (p=NS) from baseline in the MAF fatigue score in the group receiving the dipyridamole/prednisolone+DMARD combination in comparison to the placebo+DMARD group, as shown in FIG. 2A. MAF measurements taken over the course of the 42-day study for the placebo group and the combination group are shown in FIG. 2B. Mean baseline MAF values were 26.7 for placebo and 27.6 for the dipyridamole/prednisolone combination. It can be concluded that the dipyridamole/prednisolone combination tested in the study demonstrated efficacy in reducing fatigue.

### Other Embodiments

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

All publications mentioned in this specification are herein incorporated by reference to the same extent as if each independent publication was specifically and individually incorporated by reference.

What is claimed is:

1. A method for treating pain or fatigue associated with a musculoskeletal disorder, said method comprising administering to a patient diagnosed with or at risk of developing said pain or fatigue a tetra-substituted pyrimidopyrimidine and a corticosteroid, wherein said tetra-substituted pyrimidopyrimidine and said corticosteroid are administered simultaneously or within fourteen days of each other in amounts sufficient to treat said patient.

2. A method for treating tenderness, impairment in mobility, soft tissue swelling, or bony swelling associated with a musculoskeletal disorder, said method comprising administering to a patient diagnosed with or at risk of developing said tenderness, impairment in mobility, soft tissue swelling, or bony swelling a tetra-substituted pyrimidopyrimidine and a corticosteroid, wherein said tetra-substituted pyrimidopyrimidine and said corticosteroid are administered simultaneously or within fourteen days of each other in amounts sufficient to treat said patient.

3. A method for treating a Group B musculoskeletal disorder, said method comprising administering to a patient diagnosed with or at risk of developing said musculoskeletal disorder a tetra-substituted pyrimidopyrimidine and a corticosteroid, wherein said tetra-substituted pyrimidopyrimidine and said corticosteroid are administered simultaneously or within fourteen days of each other in amounts sufficient to treat said patient.

4. The method of claim 3, wherein said Group B musculoskeletal disorder is selected from the group consisting of acquired hyperostosis syndrome, acromegaly, chronic fatigue syndrome, congenital hypothyroidism, dentigerous cyst, diffuse idiopathic skeletal hyperostosis, Dupuytren's contracture, eosinophilia-myalgia syndrome, Felty's syndrome, hallux valgus, Kabuki make-up syndrome, Legg-Perthes disease, Lyme disease, Melas syndrome, neurogenic arthropathy, osteitis deformans, osteochondritis, osteomalacia, osteomyelitis, osteonecrosis, osteoporosis, Paget's disease, Pierre Robin syndrome, polymyositis, postpoliomyelitis syndrome, pseudogout, Reiter disease, renal osteodystrophy, rhabdomyolysis, Sever's disease (calceneal apophysitis), spinal stenosis, synovitis, tendinopathy, tennis elbow, tenosynovitis, and Tietze's syndrome.

5. The method of claim 1, said method further comprising administering to said patient a third drug selected from the group consisting of a corticosteroid, an NSAID, a COX-2 inhibitor, a biologic, a small molecule immunomodulator, a DMARD, a xanthine, an NsIDI, a vitamin D analog, a psoralen, a retinoid, 5-amino salicylic acid, hydroxychloroquine sulfate, and penicillamine, wherein said tetra-substituted pyrimidopyrimidine, said corticosteroid, and said third drug are administered simultaneously or within fourteen days of each other in amounts sufficient to treat said patient.

6. The method of claim 5, wherein said third drug is a corticosteroid or a DMARD.

7. The method of claim 1, wherein said patient experiences a reduction in said pain or fatigue subsequent to said treatment.

8. The method of claim 7, wherein said reduction in pain or fatigue is measured using a 10 cm visual analog scale, a Likert scale, the Lequesne index, the WOMAC index, the Piper Fatigue scale, or the Multidimensional Assessment of Fatigue scale.

9. The method of claim 8, wherein said reduction in pain or fatigue is measured using an AUSCAN index that utilizes a 10 cm visual analog scale or a Likert Scale.

10. The method of claim 1, wherein said musculoskeletal disorder is osteoarthritis.

11. The method of claim 1, wherein said tetra-substituted pyrimidopyrimidine is dipyridamole.

12. The method of claim 1, wherein said corticosteroid is prednisolone.

13. The method of claim 12, wherein said tetra-substituted pyrimidopyrimidine is dipyridamole.

14. The method of claim 1, wherein said tetra-substituted pyrimidopyrimidine and said corticosteroid are administered in the same pharmaceutical formulation.

15. The method of claim 1, wherein said tetra-substituted pyrimidopyrimidine is administered in an amount of 0.5-800 mg/day and said corticosteroid is administered in an amount of 0.1-1500 mg/day.

16. The method of claim 15, wherein said tetra-substituted pyrimidopyrimidine is administered in an amount of 18-600 mg/day and said corticosteroid is administered in an amount of 0.5-30 mg/day.

17. The method of claim 16, wherein said corticosteroid is administered in an amount of 0.5-10 mg/day.

18. The method of claim 1, wherein said tetra-substituted pyrimidopyrimidine or said corticosteroid is formulated for topical administration.

19. The method of claim 1, wherein said tetra-substituted pyrimidopyrimidine or said corticosteroid is formulated for systemic administration.

20. The method of claim 1, wherein said tetra-substituted pyrimidopyrimidine or said corticosteroid is administered in a low dosage.

21. The method of claim 1, wherein said tetra-substituted pyrimidopyrimidine or said corticosteroid is administered in a high dosage.

22. A kit comprising:
(i) a composition comprising a tetra-substituted pyrimidopyrimidine and a corticosteroid; and
(ii) instructions for administering said composition to a patient diagnosed with or at risk of developing a musculoskeletal disorder.

23. A kit comprising:
(i) a tetra-substituted pyrimidopyrimidine;
(ii) a corticosteroid; and
(iii) instructions for administering said tetra-substituted pyrimidopyrimidine and said corticosteroid to a patient diagnosed with or at risk of developing a musculoskeletal disorder.
